SEQUENCE LISTING (28 sequences)

SEQ ID NO: 1 - DNA, 22 bp, synthetic construct
  misc_feature 1..22: note = CRE Recognition sequence
  source 1..22: mol_type = other DNA
caaaacgtcgtgagacagtttg

SEQ ID NO: 2 - DNA, 3181 bp, synthetic construct
  misc_feature 1..3181: note = Constructed sequence
  repeat_region 1..168: note = 5' ITR
  regulatory 211..907: note = promoter - TBG with enhancer; regulatory_class = promoter
  regulatory 211..310: note = promoter - alpha mic/bik enhancer; regulatory_class = promoter
  regulatory 317..419: note = promoter - alpha mic/bik enhancer; regulatory_class = promoter
  regulatory 474..907: note = promoter - TBG promoter; regulatory_class = promoter
  misc_feature 939..1071: note = Intron
  misc_feature 2022..2743: note = WPRE
  regulatory 2750..2964: note = polyA_signal - BGH polyA; regulatory_class = polyA_signal_sequence
  repeat_region 3014..3181: note = 3' ITR
  source 1..3181: mol_type = other DNA
  CDS 1089..2183: note = ARCUS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - protein, 364 aa, synthetic construct
  REGION 1..364: note = Synthetic Construct
MAPKKKRKVHMNTKYNKEFLLYLAGFVDGDGSIFARIKPSQRSKFKHKLHLVFAVYQKTQRRWFLDKLVDEIGVGYVLDSGSVSFYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIKPVQRAKFKHELVLGFDVTQKTQRRWFLDKLVDEIGVGYVYDKGSVSAYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 4 - DNA, 1068 bp, synthetic construct
  misc_feature 1..1068: note = engineered nucleic acid sequence hOTCco2
  source 1..1068: mol_type = other DNA
atgctgttcaacctgcggatcctgctgaacaacgccgccttccggaacggacacaacttcatggtgcggaacttccggtgcggacagccactgcagaacaaggtgcagctgaagggacgggacctgctgaccctgaagaacttcaccggagaggagatcaagtacatgctgtggctgagcgccgatctgaagttccggatcaagcagaagggagagtacctgccactgctgcagggaaagagcctgggaatgatcttcgagaagcggagcacccggacccggctgagcaccgagaccggattcgccctgctgggaggacacccatgcttcctgaccacccaggacatccacctgggagtgaatgagagcctgaccgacaccgcccgggtgctgagcagcatggccgacgccgtgctggcccgggtgtacaagcagagcgacctggataccctggccaaggaggccagcatcccaatcatcaacggactgagcgacctgtaccacccaatccagatcctggccgattacctgaccctgcaggagcactacagcagcctgaagggactgaccctgagctggatcggagacggaaacaacatcctgcacagcatcatgatgagcgccgccaagttcggaatgcacctgcaggccgccaccccaaagggatacgagccagatgccagcgtgaccaagctggccgagcagtacgccaaggagaacggaaccaagctgctgctgaccaacgacccactggaggccgcccacggaggaaacgtgctgatcaccgatacctggatcagcatgggacaggaggaggagaagaagaagcggctgcaggccttccagggataccaggtgaccatgaagaccgccaaggtggccgccagcgattggaccttcctgcactgcctgccacggaagccagaggaggtggacgacgaggtgttctacagcccacggagcctggtgttcccagaggccgagaaccggaagtggaccatcatggccgtgatggtgagcctgctgaccgattacagcccacagctgcagaagccaaagttctgataa

SEQ ID NO: 5 - DNA, 1062 bp, Homo sapiens
  source 1..1062: mol_type = other DNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattt

SEQ ID NO: 6 - DNA, 3474 bp, synthetic construct
  misc_feature 1..3474: note = Constructed Sequence
  repeat_region 1..168: note = 5' ITR
  misc_feature 178..677: note = Left HDR arm
  regulatory 684..783: note = promoter - alpha mic/bik enhancer; regulatory_class = promoter
  regulatory 794..893: note = promoter - alpha mic/bik enhancer; regulatory_class = promoter
  regulatory 951..1384: note = promoter - TBG promoter; regulatory_class = promoter
  misc_feature 1477..2544: note = hOTCco2 coding sequence
  regulatory 2561..2775: note = polyA_signal - BGH poly A; regulatory_class = polyA_signal_sequence
  misc_feature 2782..3281: note = Right HDR Arm
  repeat_region 3307..3474: note = 3' ITR
  source 1..3474: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaaacgcgtacctaggtccccccggcctatcaaggcttccctggcggccgaatttaaaggcatcaagcaaacaaagcccaacacatctctgccttgtcctctcagtttccccccgtggcacttagaaccacttgatacaccgaatagtttccggtctatctcccccactaggatgtaaactccacaggggcattgggaatgctgcctggctatggtagggacagaggggagcaccagggcggggcaggggtgccagagttctgcctgggcagtcagattttccttaggaggggacatttgagtgggacccaaacaggtgtatagcagttgtccagcccagctggcaaggcctgagtctgcctctgcaacccctctcttgggctcctttctctgccacccacctcctcacctttccaggtcatcacagttggggccaccaatgcccaggaccagccggtgaccctggggactttggggaccaactttggccgctgtgtggacctctttgcgctagcaggttaatttttaaaaagcagtcaaaagtccaagtggcccttggcagcatttactctctctgtttgctctggttaataatctcaggagcacaaacattccacgatcatgcaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagccgccaccatgctgttcaacctgcggatcctgctgaacaacgccgccttccggaacggacacaacttcatggtgcggaacttccggtgcggacagccactgcagaacaaggtgcagctgaagggacgggacctgctgaccctgaagaacttcaccggagaggagatcaagtacatgctgtggctgagcgccgatctgaagttccggatcaagcagaagggagagtacctgccactgctgcagggaaagagcctgggaatgatcttcgagaagcggagcacccggacccggctgagcaccgagaccggattcgccctgctgggaggacacccatgcttcctgaccacccaggacatccacctgggagtgaatgagagcctgaccgacaccgcccgggtgctgagcagcatggccgacgccgtgctggcccgggtgtacaagcagagcgacctggataccctggccaaggaggccagcatcccaatcatcaacggactgagcgacctgtaccacccaatccagatcctggccgattacctgaccctgcaggagcactacagcagcctgaagggactgaccctgagctggatcggagacggaaacaacatcctgcacagcatcatgatgagcgccgccaagttcggaatgcacctgcaggccgccaccccaaagggatacgagccagatgccagcgtgaccaagctggccgagcagtacgccaaggagaacggaaccaagctgctgctgaccaacgacccactggaggccgcccacggaggaaacgtgctgatcaccgatacctggatcagcatgggacaggaggaggagaagaagaagcggctgcaggccttccagggataccaggtgaccatgaagaccgccaaggtggccgccagcgattggaccttcctgcactgcctgccacggaagccagaggaggtggacgacgaggtgttctacagcccacggagcctggtgttcccagaggccgagaaccggaagtggaccatcatggccgtgatggtgagcctgctgaccgattacagcccacagctgcagaagccaaagttctgataacggccgaggaggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacaattgttgccccaggggaggacatcattggtgcctccagcgactgcagcacctgctttgtgtcacagagtgggacatcacaggctgctgcccacgtggctggtaagtcaccaccccactgcctcggccaccgtgatgctaacagcccctttggcagtcagggtctgtgccgggacctccagtgccaggctctgtgcagggggaccagagatgaagtaggcctgatggtgccttcaaggacactcagtctgatgagggaggcgagtgcacagagggaacacgaggtcagggctgtattagagggagcccagaggaggcacctgcccagcccgagggtcagagaaggcatcttggaggagggacatttgatcgggagcttgatggatgaataggagtttacctggccgataagacagcaactaccaaggcttagaggtgtgagaggaggctgtcttacctcactgagtaaggactgcaggcggcttaccttcgagaagagagcttagtgtctgtgtgcacgtgccatggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 132 bp, Homo sapiens
  source 1..132: mol_type = other DNA
tgggctcctttctctgccacccacctcctcacctttccaggtcatcacagttggggccaccaatgcccaagaccagccggtgaccctggggactttggggaccaactttggccgctgtgtggacctctttgc

SEQ ID NO: 8 - DNA, 137 bp, Homo sapiens
  source 1..137: mol_type = other DNA
ttgccccaggggaggacatcattggtgcctccagcgactgcagcacctgctttgtgtcacagagtgggacatcacaggctgctgcccacgtggctggtaagtcaccaccccactgcctcggccaccgtgatgctaac

SEQ ID NO: 9 - DNA, 500 bp, Homo sapiens
  source 1..500: mol_type = other DNA
acctaggtccccccggcctatcaaggcttccctggcggccgaatttaaaggcatcaagcaaacaaagcccaacacatctctgccttgtcctctcagtttccccccgtggcacttagaaccacttgatacaccgaatagtttccggtctatctcccccactaggatgtaaactccacaggggcattgggaatgctgcctggctatggtagggacagaggggagcaccagggcggggcaggggtgccagagttctgcctgggcagtcagattttccttaggaggggacatttgagtgggacccaaacaggtgtatagcagttgtccagcccagctggcaaggcctgagtctgcctctgcaacccctctcttgggctcctttctctgccacccacctcctcacctttccaggtcatcacagttggggccaccaatgcccaggaccagccggtgaccctggggactttggggaccaactttggccgctgtgtggacctctttgc

SEQ ID NO: 10 - DNA, 500 bp, Homo sapiens
  source 1..500: mol_type = other DNA
ttgccccaggggaggacatcattggtgcctccagcgactgcagcacctgctttgtgtcacagagtgggacatcacaggctgctgcccacgtggctggtaagtcaccaccccactgcctcggccaccgtgatgctaacagcccctttggcagtcagggtctgtgccgggacctccagtgccaggctctgtgcagggggaccagagatgaagtaggcctgatggtgccttcaaggacactcagtctgatgagggaggcgagtgcacagagggaacacgaggtcagggctgtattagagggagcccagaggaggcacctgcccagcccgagggtcagagaaggcatcttggaggagggacatttgatcgggagcttgatggatgaataggagtttacctggccgataagacagcaactaccaaggcttagaggtgtgagaggaggctgtcttacctcactgagtaaggactgcaggcggcttaccttcgagaagagagcttag

SEQ ID NO: 11 - DNA, 516 bp, Macaca mulatta
  source 1..516: mol_type = other DNA
aagcatacctgacagcggtaacccagatcctccctggcctcggaggcttccctggcagcccaatttaaaggcatcaagcaaacaaagcccaacacatctctgccttgccctctcagtctccccccgtggcacttagagccacctgatacaccgagtagtttcctatctccctcactagaatgtaaactccacaggggcattgggaatgctgcctggctatggtagggacagaggggagcaccagggcggggcaggggtgccagagttctgcctggggagtcagattttccttaggaggggacatttgaatgggacccaaacaggtgtatagcagttgttcagcccagctggcaaggcctcagtttgcttctgcaacccctctcttgggctcctttctgtgccacctacctcctcacctttccaggtcatcacagttggggccaccaatgcccaggaccagccggtgaccctggggactttggggaccaactttggccgctgtgtggacctctttgc

SEQ ID NO: 12 - DNA, 510 bp, Macaca mulatta
  source 1..510: mol_type = other DNA
ttgccccaggggaggacatcattggtgcctccagcgactgcagcacctgctttgtgtcacggagtgggacatcgcaggctgctgcccacgtggctggtaagtcaccaccccactgccttggccaccgtgatgctaacagccctttggcggtcagggtctgtgccaggacctccagtgccaggctctgtgcagggggaccagagatgagacaggcctgatggtgccttcatggacactcagtctgatgagggagacgagtgcacagtgggaacacgaggttagggctgtattagagggagcccagaggaggcacctgcccagcccgagggtcagagaaggcttcttggacaagggacatttgatctggagcttgatggatgaataggagtccacctggccgataagacagcaactaccaaggcttagaggtgtgacggaaggctgtcttacctcactgtgtgagggactgcaggcggcttaccttctagaagagagcttggtgtctctggt

SEQ ID NO: 13 - DNA, 500 bp, Homo sapiens; identical to SEQ ID NO: 9
  source 1..500: mol_type = other DNA

SEQ ID NO: 14 - DNA, 500 bp, Homo sapiens; identical to SEQ ID NO: 10
  source 1..500: mol_type = other DNA

SEQ ID NO: 15 - DNA, 2214 bp, Adeno-associated Virus rh79
  source 1..2214: mol_type = other DNA
  CDS 1..2214
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgatcccccgactcctccacgggcatcggcaaaaaaggccagcagcccgcgagaaagagactgaactttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaatgggacatcgggaggaagcaccaacgacaacacctacttcggctacagcaccccctgggggtattttgacttcaacagattccactgtcacttctcaccacgtgactggcagagactcatcaacaacaactggggattccggcccaagagactcagcttcaagctcttcaacatccaggttaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaacggaagtcaagccgtaggccgttcctcattctactgcctggaatattttccatctcaaatgctgcggactggaaacaactttgaatttagctacacctttgaggacgtgcccttccacagcagctacgcacacagccagagcctggaccggctgatgaaccctctcatcgaccagtacctgtattacctatccagaactcagtccacaggaggaactcaaggtacacagcaattgttattttctcaagccgggcctgcaaatatgtcggctcaggccaagaactggctacctggaccttgctaccggcagcagcgagtctccacgacactgtcgcaaaacaacaacagcaactttgcttggactggtgccacgaaatatcatctgaacggaagagactctttggtgaatcccggtgttgctatggcaacgcataaggacgacgaggaacgtttctttccatcgagcggagtcctgatgtttggaaaacagggtgctggaagagacaatgtggactatagcagcgttatgctaaccagcgaggaagaaatcaagaccactaaccctgtagccactgaacaatacggcgtggtggctgataacttgcagcaaaccaatacaggacctatcgtgggaaatgtcaacagccaaggagccttacctggcatggtctggcagaaccgagacgtgtacctgcagggtcccatttgggccaagattcctcacacggacggcaactttcacccgtctcctctgatgggcggctttggactgaaacacccgcctcctcaaatcctgatcaagaacactcccgttcctgcggatcctccaacgacgttcagccaggcgaaattggcttccttcatcacgcagtatagtaccggccaggtcagcgtggagatcgagtgggagctgcagaaggagaacagcaagcgctggaacccagaaattcagtatacttccaactactacaaatctacaaatgtggactttgctgtcaataccgagggtacatattcagagcctcgccccattggaactcgttacctcacccgtaatctg

SEQ ID NO: 16 - protein, 738 aa, Adeno-associated Virus rh79
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 17 - DNA, 265 bp, Homo sapiens
  source 1..265: mol_type = other DNA
  misc_feature 121..141: note = ARCUS targeting sequence
tgggctcctttctctgccacccacctcctcacctttccaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggtaagtcaccaccccactgcctcggccaccgtgatgctaac

SEQ ID NO: 18 - DNA, 22 bp, synthetic construct
  source 1..22: mol_type = other DNA
tggacctctttgccccagggga

SEQ ID NO: 19 - DNA, 22 bp, synthetic construct
  source 1..22: mol_type = other DNA
tggcccttggtgccacagggaa

SEQ ID NO: 20 - DNA, 22 bp, synthetic construct
  source 1..22: mol_type = other DNA
aggaccttggtgagcccgggaa

SEQ ID NO: 21 - DNA, 22 bp, synthetic construct
  source 1..22: mol_type = other DNA
gggccctccttgccccagggaa

SEQ ID NO: 22 - DNA, 22 bp, synthetic construct
  source 1..22: mol_type = other DNA
tggtctgtggtaccccagggga

SEQ ID NO: 23 - DNA, 22 bp, synthetic construct
  source 1..22: mol_type = other DNA
tggccccttttacaggagggcc

SEQ ID NO: 24 - DNA, 22 bp, synthetic construct
  source 1..22: mol_type = other DNA
tggccctcttttccagaggcca

SEQ ID NO: 25 - DNA, 22 bp, synthetic construct
  source 1..22: mol_type = other DNA
tcgaccctggtaccccagggcg

SEQ ID NO: 26 - DNA, 22 bp, synthetic construct
  source 1..22: mol_type = other DNA
tagacctcttttccccagggtc

SEQ ID NO: 27 - DNA, 22 bp, synthetic construct
  source 1..22: mol_type = other DNA
tgtccctctgtgccccaggcca

SEQ ID NO: 28 - DNA, 22 bp, synthetic construct
  source 1..22: mol_type = other DNA
tgtccctctgtcccccaggcta

(19) United States
(12) Patent Application Publication
(10) Pub. No.: US 2025/0161493 A1
(43) Pub. Date: May 22, 2025

(54) COMPOSITIONS AND METHODS FOR IN VIVO NUCLEASE-MEDIATED TREATMENT OF ORNITHINE TRANSCARBAMYLASE (OTC) DEFICIENCY

(71) Applicant: The Trustees of the University of Pennsylvania, Philadelphia, PA (US)

(72) Inventors: Lili Wang, Phoenixville, PA (US); James M. Wilson, Philadelphia, PA (US); Anna Tretiakova, Philadelphia, PA (US)

(73) Assignee: The Trustees of the University of Pennsylvania, Philadelphia, PA (US)

(21) Appl. No.: 18/726,190

(22) PCT Filed: Nov. 1, 2022
(86) PCT No.: PCT/US2022/079020

§ 371 Date: Jul. 2, 2024

Related U.S. Application Data
(60) Provisional application No. 63/379,067, filed on Oct. 11, 2022.
(60) Provisional application No. 63/370,049, filed on Aug. 1, 2022.
(60) Provisional application No. 63/364,861, filed on May 17, 2022.
(60) Provisional application No. 63/331,384, filed on Apr. 15, 2022.
(60) Provisional application No. 63/301,917, filed on Jan. 21, 2022.

(51) Int. Cl. A61K 48/00; A61K 9/00; A61K 38/45; A61P 3/00; C12N 9/22; C12N 15/86; C12N 15/90

(52) U.S. Cl. CPC A61K 48/0058; A61K 9/0019; A61K 38/45; A61K 48/0075; A61K 48/0083; A61P 3/00; C12N 9/22; C12N 15/86; C12N 15/907; C12Y 201/03003; C12N 2750/14143; C12N 2800/40

(57) ABSTRACT

A dual vector system for treating ornithine transcarbamylase deficiency is provided. The system includes ((a) a gene editing AAV comprising a first AAV rh79 capsid and a first vector genome comprising a 5′ ITR, a sequence encoding a meganuclease that targets PCSK9 under control of regulatory sequences that direct expression of the meganuclease in a target cell comprising a PCSK9 gene, and a 3′ ITR; and
(b) a donor AAV vector comprising a second AAV capsid and a second AAV rh79 vector genome comprising: a 5′ITR, a 5′ homology directed recombination (HDR) arm, a transgene encoding ornithine transcarbamylase (OTC) and regulatory sequences that direct expression of the transgene in the target cell, a 3′ HDR arm, and a 3′ ITR.

### BACKGROUND OF THE INVENTION

Ornithine transcarbamylase (OTC) deficiency (OTCD) is an X-linked urea cycle disorder associated with high mortality. Although a promising treatment for late-onset OTC deficiency, adeno-associated virus (AAV) neonatal gene therapy would only provide short-term therapeutic effects as the non-integrated genome gets lost during hepatocyte proliferation.

Meganucleases generate double strand breaks (DSBs) in the chromosome, leading to DNA repair. In the presence of donor DNA, homology directed repair (HDR) occurs and replaces genetic information in the chromosome with new information from the donor gene.

Safe harbor sites (SHS) are genomic loci where genes or other genetic elements can be safely inserted and expressed. These SHS are critical for effective human disease gene therapies; for investigating gene structure, function and regulation; and for cell marking and tracking.

Nuclease-mediated, site-specific integration of a transgene cassette in a safe harbor in the genome would provide long-term therapeutic benefits to patients with OTC deficiency.

What are needed are improved compositions and methods for treatment of OTC.

### SUMMARY OF THE INVENTION

Provided herein are compositions, methods, systems, and kits for treatment of OTC in a subject in need thereof, which allow knockdown or ablation of the native PCSK9 gene and insertion and/or expression of an exogenous OTC transgene in the PCSK9 gene locus.

In one aspect, a dual vector system for treating an ornithine transcarbamylase deficiency is provided. The system includes (a) a gene editing AAV comprising a first AAV capsid and a first vector genome comprising a 5′ ITR, a sequence encoding a meganuclease that targets PCSK9 under control of regulatory sequences that direct expression of the meganuclease in a target cell comprising a PCSK9 gene, and a 3′ ITR; and (b) a donor AAV vector comprising a second AAV capsid and a second vector genome comprising: a 5′ITR, a 5′ homology directed recombination (HDR) arm, a transgene encoding ornithine transcarbamylase (OTC) and regulatory sequences that direct expression of the transgene in the target cell, a 3′ HDR arm, and a 3′ ITR. In certain embodiments, the meganuclease is the ARCUS meganuclease having the sequence of SEQ ID NO: 3. In certain embodiments, the sequence encoding a meganuclease comprises nucleotides (nt) 1089-2183 of SEQ ID NO: 2, or a sequence at least 90% identical to nucleotides (nt) 1089-2183 of SEQ ID NO: 2. In certain embodiments, the transgene encoding OTC comprises SEQ ID NO: 5, or a sequence at least 90% identical to SEQ ID NO: 5. In certain embodiments, the first and second AAV capsid are AAVrh79 capsids of SEQ ID NO: 16.

In another aspect, a method of treating an OTC deficiency in a subject in need thereof is provided. The method includes co-administering to the subject having OTC (a) a gene editing AAV comprising a first AAV capsid and a first vector genome comprising a 5′ ITR, a sequence encoding a meganuclease that targets PCSK9 under control of regulatory sequences that direct expression of the meganuclease in a target cell comprising a PCSK9 gene, and a 3′ ITR; and (b) a donor AAV vector comprising a second AAV capsid and a second vector genome comprising: a 5′ITR, a 5′ homology directed recombination (HDR) arm, a transgene encoding ornithine transcarbamylase (OTC) and regulatory sequences that direct expression of the transgene in the target cell, a 3′ HDR arm, and a 3′ ITR. In certain embodiments, i) the first vector genome comprises nt 211 to 2964 of SEQ ID NO: 2, or a sequence sharing at least 90% identity with nt 211 to 2964 of SEQ ID NO: 2; and ii) the second vector genome comprises nt 178 to 3281 of SEQ ID NO: 6 or a sequence sharing at least 90% identity with nt 178 to 3281 of SEQ ID NO: 6.

Other aspects and advantages of the invention will be apparent from the following detailed description of the invention.

### BRIEF DESCRIPTION OF THE DRAWINGS

FIG. 1A shows a timeline for a study comprising an hOTC mini-gene knock-in in PCSK9 locus by ARCUS2. FIG. 1B shows study design for Groups (G) 1-7. Animals 21-111, 21-122, and 21-113 were AAV binding antibody (BAb) positive prior to dosing.

FIG. 2 shows a schematic representation for a dual AAV vector system for ARCUS2-mediated gene correction, wherein the AAV-donor vector comprises an hOTC donor template sequence, as used in the study shown in FIG. 1A-1B. Different HDR arms are used, as shown.

FIG. 3 is a chart showing experimental results of experiment as described in FIG. 1A-2. Details are provided in FIGS. 4A-4K.

FIGS. 4A-4K show the results of experiment described in FIGS. 1A-2. FIG. 4A shows PCSK9 levels shown as % of day 0 for the groups. FIG. 4B shows ALT levels shown as U/L for the groups. Liver biopsies were performed at noted time points and dual in situ hybridization (ISH) using specific probes to detect hOTC and ARCUS was performed. FIG. 4C shows transduction efficiency of OTC transgene as quantified by ISH, and plotted as percent hepatocytes transduced. FIG. 4D shows transduction efficiency of OTC transgene as quantified by IF. FIG. 4E shows body weight of NHP. FIG. 4F shows vector GCs in liver by quantitative PCR analysis. FIG. 4G shows expression of hOTC and nuclease in macaque liver measured by quantitative PCR on total RNA isolated from the liver biopsy samples followed by reverse transcription and presented as relative expression levels normalized by GAPDH levels. FIG. 4H shows Indel analysis on the rhPCSK9-targeted locus performed by amplicon-seq at the indicated time points. FIG. 4I shows on-target indels in the noted tissues. The only non-liver tissue with indels was pancreas. FIG. 4J shows a comparison between LFTs (IU/mL) of newborn and infant NHPs treated with 4×10¹³ GC/kg versus adults treated with AAV.Arcus only. FIG. 4K shows OTC enzyme activity staining at 1-year necropsy in some Group 2 and Group 3 animals. Abbreviations: GAPDH, glyceraldehyde-3-phosphate dehydrogenase; GC, genome copies; hOTC, human ornithine transcarbamylase; OT, off-target; PCR, polymerase chain reaction; rhPCSK9, proprotein convertase subtilisin/kexin type 9 (rhesus gene); RNA, ribonucleic acid.

FIGS. 5A and 5B show schematic representation of a PCSK9-hE7-KI Mouse Model. FIG. 5A shows schematic representation of the mouse pcsk9 exon 7 which is replaced with human pcsk9 exon 7 (hE7 contains ARCUS targeting sequence). FIG. 5B shows schematic representation of crossing PCSK9-hE7-KI mouse model with other disease mouse models, such as OTC spf^{ash}, the KI-spf^{ash} model. The PCSK9-hE7-KI knock-in mouse model was first generated by replacing a region including exon 7 of the murine Pcsk9 gene with a region of human PCSK9 gene containing exon 7. The PCSK9-hE7-KI mouse was then crossed with sparse fur ash (spf^{ash}) mouse, which exhibits a 20-fold reduction in OTC expression due to a G to A point mutation at the splice donor site at the end of exon 4 of the Otc gene. The mice from this cross were termed PCSK9-hE7-KI.spf^{ash} mice and were utilized as described herein. Abbreviations: bp, base pairs; E6, exon 6; E7: exon 7; E8, exon 8; HDR, homology-dependent recombination; PCSK9, proprotein convertase subtilisin/kexin type 9 (gene, human); Pcsk9, proprotein convertase subtilisin/kexin type 9 (gene, mouse). FIG. 5C shows the sequence of the human exon 7 region and part of the adjacent intron sequence swapped in the murine Pcsk9 locus (SEQ ID NO: 17).

FIG. 6 shows sequence alignment of 265 bp sequence that represents the human PCSK9 sequence of the pcsk9-hE7 knock-in allele, mouse PCSK9 (mPCSK9) and rhesus macaques PCSK9 (rhPCSK9).

FIG. 7 shows a schematic representation donor construct for a dual AAV vector system for ARCUS2-mediated gene correction, wherein the AAV-donor vector comprises an hOTC donor template sequence. The homology of the HDR arms in the constructs with the knock in mouse model (FIG. 5), NHP, and human target regions is shown.

FIG. 8A shows a timeline for a study comprising an hOTC mini-gene knock-in in PCSK9 locus by ARCUS2 performed in PCSK9-hE7-KI.spf-ash pups (partial OTC deficiency model).

FIG. 8B shows the vectors and dosages each group will receive for the study of FIG. 8A.

FIGS. 9A-9F show results of the study of mice shown in FIG. 8A-8B, treated with vectors as shown in FIG. 7, or untreated (KI WT) and fed a high protein (HP) diet for 10 days. FIG. 9A shows probability of survival. FIG. 9B shows weight as a percentage of weight prior to introduction of the HP diet. FIG. 9C shows plasma NH₃ levels at day 10 of HP diet. FIG. 9D shows mPCSK9 protein levels at day 48. FIG. 9E shows indel % as measured by amplicon-seq on day 59. FIG. 9F shows vector transduction levels in liver biopsy samples, plotted as AAV genome copies (GC) per diploid cell, measured on day 59.

FIG. 10 is a schematic of a two-vector approach for treatment of OTC deficiency. Both vectors use a clade E capsid, AAVrh79, and the liver-specific TBG promoter. The first vector is the nuclease, ARCUS, and the second is the hOTC donor gene cassette flanked by 500 bp arms of homology for PCSK9 exon 7.

FIG. 11 is a plasmid map for the donor construct for the two-vector approach described in FIG. 10. The sequence of the plasmid from ITR to ITR is shown in SEQ ID NO: 6.

FIG. 12 is a plasmid map for the nuclease construct for the two-vector approach described in FIG. 10. The sequence of the plasmid from ITR to ITR is shown in SEQ ID NO: 2.

FIG. 13 is a table showing exemplary HDR sequences used in donor constructs provided herein.

FIGS. 14A-14B show the results of amplicon-seq validation of off target editing for the experiment as described for FIGS. 4A-4K. FIG. 14A provides a list of the off-target sites, along with the chromosomal location and best match to off-target consensus sequence. FIG. 14B is a graph showing indel percentages for OT1-OT10. Editing on OT1, OT4, and OT5 were significantly higher in ARCUS+donor animals than in non-nuclease controls.

FIG. 15A shows a timeline for a MED study comprising an hOTC mini-gene knock-in in PCSK9 locus by ARCUS2 performed in PCSK9-hE7-KI.spf-ash pups, as discussed in Example 7.

FIG. 15B shows the study design for the study shown in FIG. 15A.

FIG. 16 shows the partial study design for a 1 year toxicity study performed in NHP, as discussed in Example 9.

### DETAILED DESCRIPTION OF THE INVENTION

Provided herein are compositions, kits, and methods which provide stable, long term therapeutic effects to patients with certain genetic disorders, including liver metabolic disorders. The compositions, kits, and methods utilize a nuclease that targets the PCSK9 locus of the target cell, and a donor vector provides a template which includes an exogenous product for integration into, and expression from, the PCSK9 locus, wherein the inserted nucleic acid sequence does not encode PCSK9, and the expression of the endogenous PCSK9 is disrupted and expression levels are reduced.

In one embodiment, the test article described herein is comprised of 2 vectors both using a clade E capsid, AAVrh79, and the liver-specific TBG promoter. The first vector is the nuclease, ARCUS, and the second is the hOTC donor gene cassette flanked by 500 bp arms of homology for PCSK9 exon 7.

In Pcsk9-hE7-KI.spf-ash mice, the test article decreased mPCSK9 levels and improved both weight loss and survival following high protein diet challenge. hOTC was well transduced and resulted in good % Indels achieved compared to untreated and GFP control treated mice.

In newborn nonhuman primates, the test article resulted in 18.6% and 11.9% hOTC transduction assessed in d84 liver biopsies, both higher than the threshold for substantially benefitting patients, which is ˜5% OTC-expressing cells.

No test-article related findings were observed and ALT and PCSK9 levels (as a percentage of day 0) remained low and stable for at least 3 months post-vector administration.

In certain embodiments, the test article is comprised of two non-replicating recombinant adeno associated virus (AAV) rh79 vectors: AAVrh79.TBG.M2PCSK9.WPRE.bGH (nuclease vector) and AAVrh79.hHDR.TBG.hOTCco.bGH (donor vector), which are mixed at a ratio determined by genome copies (GC) just before dosing. As per nonclinical studies, the ratio may be a 1:3 ratio of nuclease vector and donor vector. The test article is, in certain embodiments, administered as a single dose given as intravenous (IV) infusion and dose administered is based on GC/kg of the subject's body weight.

### PCSK9

Proprotein convertase subtilisin kexin 9 (PCSK9) is a serine protease that reduces both hepatic and extrahepatic low-density lipoprotein (LDL) receptor (LDLR; 606945) levels and increases plasma LDL cholesterol. PCSK9 is critical in the regulation of plasma cholesterol homeostasis. PCSK9 binds to the low-density lipid receptor family members low density lipoprotein receptor (LDLR), very low-density lipoprotein receptor (VLDLR), apolipoprotein E receptor (LRP1/APOER) and apolipoprotein receptor 2 (LRP8/APOER2), and promotes their degradation in intracellular acidic compartments.

While the PCSK9 gene has been targeted for treatment of cholesterol related diseases, it is demonstrated herein that the PSCK9 gene locus is a safe harbor for gene targeting for insertion of other, non-PCSK9 transgenes. Thus, the compositions, kits, and methods provided herein utilize nucleases which target the PCSK9 gene locus, and insert a therapeutic transgene into the target PCSK9 locus, using a donor template.

The compositions, kits, and methods provided herein include a gene editing vector, and a donor vector which provides the therapeutic OTC transgene to be expressed in the host cell.

### Gene Editing Component

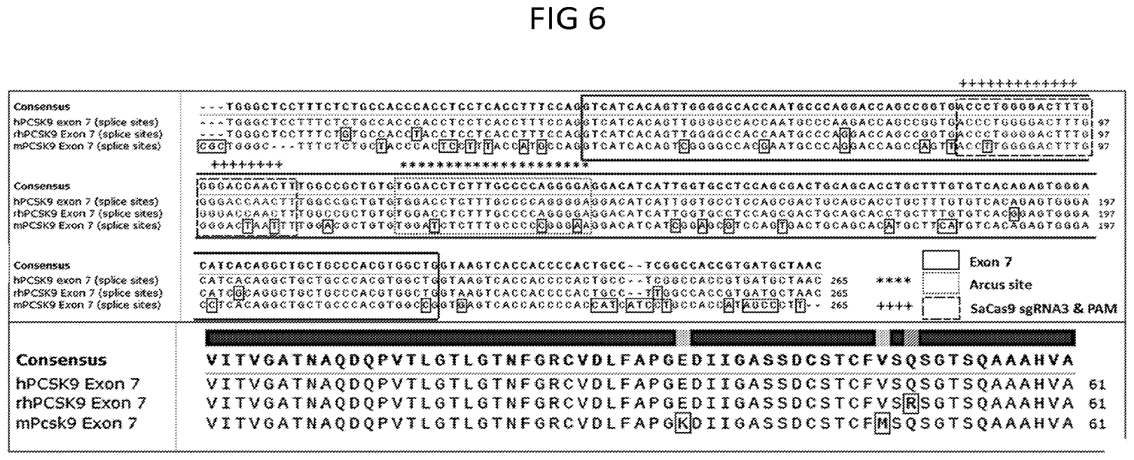

The compositions, kits, and methods provided herein include a gene editing component that comprises a nuclease (or the coding sequence therefore) and sequences which direct the nuclease to specifically target the native PCSK9 gene locus on chromosome 1. As used herein, the “target PCSK9 locus” or “PCSK9 gene locus” is in Exon 7 of the PCSK9 coding sequence. FIG. 6 provides an alignment of the human (h), rhesus (rh), and mouse (m) PCSK9 exon 7 splice sites which are exemplified herein using a SaCas9 and a meganuclease targeted to PCSK9 (referred to as ARCUS).

Described herein are compositions, particularly nucleases, which are useful targeting a gene for the insertion of a transgene, for example, nucleases that are specific for PCSK9. In certain embodiments, the nuclease is a meganuclease that targets PCSK9. Meganucleases are endodeoxyribonucleases characterized by a large recognition site (double-stranded DNA sequences of 12 to 40 base pairs), for example, I-SceI. When combined with a nuclease, DNA can be cut at a specific location. The restriction enzymes can be introduced into cells, for use in gene editing or for genome editing in situ. In certain embodiments, the nuclease is a member of the I-CreI family of homing endonucleases which recognizes and cuts a 22 base pair recognition sequence SEQ ID NO: 1—CAAAACGTCGTGAGACAGTTTG. See, e.g., WO 2009/059195. In one embodiment, the nuclease is encoded by the sequence shown in SEQ ID NO: 2, nt 1089 to 2183, or a sequence sharing at least 95%, 98%, or 99% identity thereto. In one embodiment, the nuclease protein sequence is the sequence shown in SEQ ID NO: 3. Such nuclease is sometimes referred to herein as the ARCUS nuclease. The term “homing endonuclease” is synonymous with the term “meganuclease.” See, WO 2018/195449, describing certain PCSK9 meganucleases, which is incorporated herein in its entirety.

In certain embodiments, the compositions, kits, and methods the nuclease coding sequence is comprised in a gene editing vector. The gene editing vector includes an expression cassette comprising a nucleic acid sequence encoding a nuclease and regulatory sequences that direct expression of the nuclease in a target cell comprising a PCSK9 gene. A “vector” as used herein is a biological or chemical moiety comprising a nucleic acid sequence which can be introduced into an appropriate host cell for replication or expression of said nucleic acid sequence. The vector comprising the nuclease coding sequence is an Adeno-Associated Virus (AAV) vector.

As used herein, an “expression cassette” refers to a nucleic acid molecule which comprises a biologically useful nucleic acid sequence (e.g., a gene cDNA encoding a protein, enzyme or other useful gene product, mRNA, etc) and regulatory sequences operably linked thereto which direct or modulate transcription, translation, and/or expression of the nucleic acid sequence and its gene product. As used herein, “operably linked” sequences include both regulatory sequences that are contiguous with the nucleic acid sequence and regulatory sequences that act in trans or at a distance to control the sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc, and one or more of an enhancer, or regulatory sequences downstream (3′ to) a gene sequence, e.g., 3′ untranslated region comprising a polyadenylation site, among other elements. In other embodiments, the term “transgene” refers to one or more DNA sequences from an exogenous source which are inserted into a target cell. Typically, such an expression cassette for generating a viral vector contains the coding sequence for the gene product described herein flanked by packaging signals of the viral genome and other expression control sequences such as those described herein.

In addition to the coding sequence for the nuclease, the gene editing vector includes regulatory sequences which direct expression of the nuclease in a host cell. The regulatory elements include a promoter, e.g., the liver-specific promoter thyroxin binding globulin (TBG) promoter. In certain embodiments, the TBG promoter has the sequence of nucleotides 211 to 907 of SEQ ID NO: 2, which includes enhancer sequences.

In addition to a promoter, the gene editing cassette, expression cassette and/or vector may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. In certain embodiments, the vector includes a bovine growth hormone (bGH) polyA, e.g., such as that shown in nucleotides 2750 to 2964 of SEQ ID NO: 2. A suitable enhancer includes the alpha1-microglobulin/bikunin enhancer. A suitable WPRE includes that shown in nucleotides 2202 to 2743 of SEQ ID NO: 2. These control sequences or the regulatory sequences are operably linked to the nuclease coding sequence or transgene coding sequence. In certain embodiments, a SV40 intron is included, such as that shown in nucleotides 939 to 1071 of SEQ ID NO: 2.

In certain embodiments, the nuclease vector genome includes the following components. Inverted Terminal Repeat (ITR): The ITRs are identical, reverse complementary sequences derived from AAV2 (145 base pairs [bp], GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging. Human Thyroxine-Binding Globulin (TBG) Promoter: This regulatory element confers tissue specific transgene expression in liver (410 bp, GenBank: L13470.1). Coding Sequence: The transgene is an engineered meganuclease (ARCUS; 1095 bp, 365 amino acids). It is derived from a variant of a homing endonuclease, I-CreI, isolated from Chlamydomonas reinhardtii, that recognizes and edits PCSK9 gene with high efficiency and specificity. WPRE (Woodchuck Hepatitis Virus Post-Transcriptional Regulatory Element): A cis-acting RNA element derived from the Woodchuck Hepatitis Virus (WHV) (GenBank: MT612432.1) has been inserted in the 3′ untranslated region of the coding sequence upstream of the PolyA signal. The WPRE is a hepadnavirus-derived sequence and has been previously used as a cis-acting regulatory module in viral gene vectors to achieve sufficient levels of transgene product expression and to improve the viral titers during manufacturing. The WPRE is believed to increase transgene product expression by improving transcript termination and enhancing 3′ end transcript processing, thereby increasing the amount of polyadenylated transcripts and the size of the PolyA tail and resulting in more transgene mRNA available for translation. The WPRE included in the vector is a mutated version containing 5 point mutations in the putative promoter region of the woodchuck hepatitis virus X protein (WHX) protein open reading frame (ORF), along with an additional point mutation in the start codon of the WHX protein ORF (ATG mutated to TTG). This mutant WPRE (termed mut6) is considered sufficient to eliminate expression of truncated WHX protein based on sensitive flow cytometry analyses of various human cell lines transduced with lentivirus containing a WPRE mut6-GFP fusion construct (Zanta-Boussif et al., 2009). Bovine Growth Hormone Poly A (bGH PolyA): The bGH PolyA signal (208 bp, GenBank: MT267334) facilitates efficient polyadenylation of the transgene mRNA in cis. This element functions as a signal for transcriptional termination, a specific cleavage event at the 3′ end of the nascent transcript and the addition of a long polyadenyl tail.

In certain embodiments, the gene editing vector further includes one or more nuclear localization signal (NLSs). See, e.g., Lu et al. Types of nuclear localization signals and mechanisms of protein import into the nucleus, Cell Commun Signal (May 2021) 19:60. In one embodiment, the vector contains the NLS shown in nt 1095 to 1115 of SEQ ID NO: 2.

### Donor Vector

The compositions, kits, and methods include a donor vector, which provides the coding sequence for the OTC therapeutic transgene. The donor vector contains an expression cassette comprising a nucleic acid sequence encoding a transgene, and regulatory sequences that direct expression of the transgene in the target cell. The compositions, kits, and methods for nuclease-mediated, site-specific integration of an OTC transgene cassette in a PCSK9 safe harbor in the genome that provides long-term therapeutic benefits to patients with OTC deficiency. An engineered, coding sequence for OTC, referred to herein as hOTCco2, and shown in SEQ ID NO: 4 is provided. Nucleic acids having the sequence of SEQ ID NO: 4 or sequences sharing at least 80%, at least 85%, at least 90%, at least 95%, at least 96%, at least 97%, at least 98%, at least 99%, or at least 99.9% identity with SEQ ID NO: 4 are provided. In one embodiment, the nucleic acid shares less than 80%, less than 79%, less than 78%, less than 77%, less than 76%, less than 75%, less than 74%, less than 73%, less than 72%, less than 71%, or less than 70% identity with the native OTC coding sequence that is shown in SEQ ID NO: 5.

In some embodiments, the transgene cassette includes a TBG promoter, the transgene coding sequence, and a poly A sequence.

In addition to a promoter, the transgene cassette, expression cassette and/or vector (editing or donor) may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequences or transgene coding sequence.

In certain embodiments, the donor vector genome includes the following: Inverted Terminal Repeat (ITR): The ITRs are identical, reverse complementary sequences derived from AAV2 (145 bp, GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging. 5′ and 3′ Homology Arms: Homology-dependent recombination arms (also referred to as hHDR) consisting of sequences flanking the cleavage site in exon 7 of the endoenous human PCSK9 gene locus. The homology arms comprise the sequence 500 bp upstream (5′ homology arm) and 500 bp downstream (3′ homology arm) of the ARCUS meganuclease cleavage site in exon 7 of the PCSK9 gene. Human Thyroxine-Binding Globulin (TBG) Promoter: This regulatory element confers tissue-specific transgene expression in liver (434 bp, GenBank: L13470.1). Coding Sequence: The transgene is a codon-optimized version of the human omithine transcarbamylase (OTC) gene (1068 bp, 356 amino acids). Bovine growth hormone poly A (BGH PolyA): The bGH PolyA signal (215 bp, GenBank: MT267334) facilitates efficient polyadenylation of the transgene mRNA in cis. This element functions as a signal for transcriptional termination, a specific cleavage event at the 3′ end of the nascent transcript and the addition of a long polyadenyl tail.

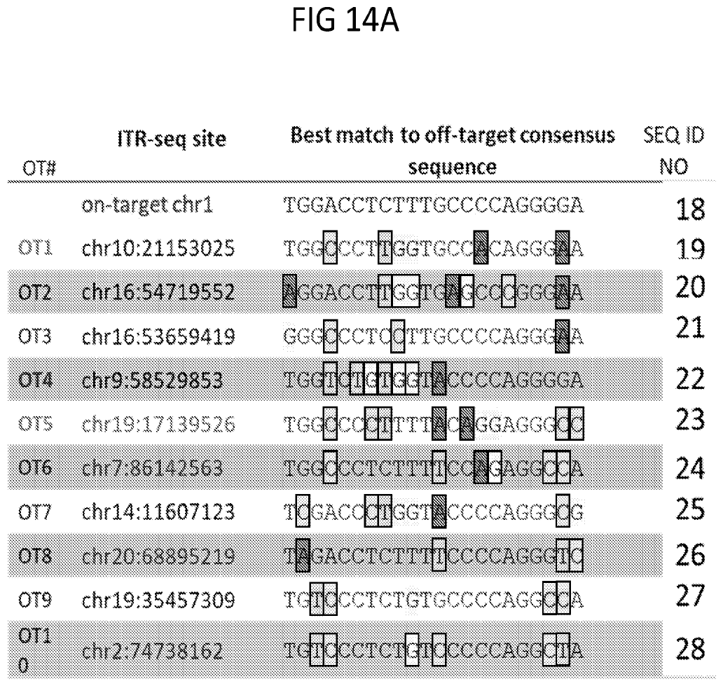

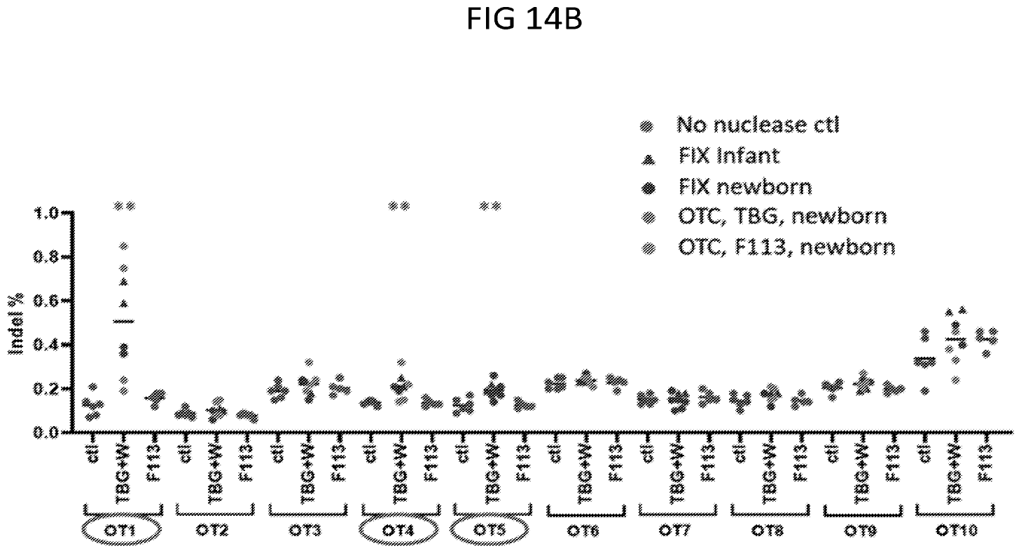

In addition to the transgene cassette, the donor vector also includes homology-directed recombination (HDR) arms 5′ and 3′ to the transgene cassette, to facilitate homology directed recombination of the transgene into the endogenous genome. The homology arms are directed to the target PCSK9 locus and can be of varying length. In another embodiment, the HDR arms are about 500 bp. The HDR arms ideally share a high level of complementarity with the target PCSK9 locus, although it need not be 100% complementarity. In some embodiments, 1, 2, 3, 4, 5, 6, 7, 8, 9, 10, 11, 12, 13, 14, 15, 16, 17, 18, 19, 20, or more mismatches are permitted in each HDR arm. Suitable HDR arm sequences, for targeting PCSK9 exon 7 are shown in FIG. 14, and SEQ ID NOs: 7-12. In one embodiment, the HDR arm sequences are those shown in SEQ ID NO: 13 and 14.

### AAV Viral Vectors

The gene editing vector and the donor vector are provided as a recombinant AAV. A “recombinant AAV” or “rAAV” is a DNAse-resistant viral particle containing two elements, an AAV capsid and a vector genome containing at least non-AAV coding sequence packaged within the AAV capsid. Unless otherwise specified, this term may be used interchangeably with the phrase “rAAV vector” or “AAV vecor”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV.

The expression cassette is located in a vector genome for packaging into a viral capsid. For example, for an AAV vector genome, the components of the expression cassette are flanked at the extreme 5′ end and the extreme 3′ end by AAV inverted terminal repeat sequences. For example, a 5′ AAV ITR, expression cassette, 3′ AAV ITR.

The source of the AAV capsid for both the donor and nuclease vectors is AAVrh79, as described in WO 2019/169004, published Sep. 6, 2019, which is incorporated herein by reference. In one embodiment, an AAVrh79 capsid comprises a heterogeneous population of AAVrh79 vp1 proteins, AAVrh79 vp2 proteins, and AAVrh79 vp3 proteins. In one embodiment, the AAVrh79 capsid is produced by expression from a nucleic acid sequence which encodes the predicted amino acid sequence of 1 to 738 of SEQ ID NO: 16. Optionally, sequences co-expressing the vp3 protein from a nucleic acid sequence excluding the vp1-unique region (about aa 1 to 137) or the vp2-unique region (about aa 1 to 203), vp1 proteins produced from SEQ ID NO: 15, or vp1 proteins produced from a nucleic acid sequence at least 70% identical to SEQ ID NO: 15 which encodes the predicted amino acid sequence of 1 to 738 of SEQ ID NO: 16. In other embodiments, the AAVrh79 vp2 proteins produced by expression from a nucleic acid sequence which encodes the predicted amino acid sequence of at least about amino acids 138 to 738 of SEQ ID NO: 16, vp2 proteins produced from a sequence comprising at least nucleotides 412 to 2214 of SEQ ID NO: 15, or vp2 proteins produced from a nucleic acid sequence at least 70% identical to at least nucleotides 412 to 2214 of SEQ ID NO: 15 which encodes the predicted amino acid sequence of at least about amino acids 138 to 738 of SEQ ID NO: 16, AAVrh79 vp3 proteins produced by expression from a nucleic acid sequence which encodes the predicted amino acid sequence of at least about amino acids 204 to 738 of SEQ ID NO: 16, vp3 proteins produced from a sequence comprising at least nucleotides 610 to 2214 of SEQ ID NO: 15, or vp3 proteins produced from a nucleic acid sequence at least 70% identical to at least nucleotides 610 to 2214 of SEQ ID NO: 15 which encodes the predicted amino acid sequence of at least about amino acids 204 to 738 of SEQ ID NO: 16.

In certain embodiments, an AAVrh79 capsid comprises: a heterogeneous population of vp1 proteins which are the product of a nucleic acid sequence encoding the amino acid sequence of SEQ ID NO: 16, a heterogeneous population of vp2 proteins which are the product of a nucleic acid sequence encoding the amino acid sequence of at least about amino acids 138 to 738 of SEQ ID NO: 16, and a heterogeneous population of vp3 proteins which are the product of a nucleic acid sequence encoding at least amino acids 204 to 738 of SEQ ID NO: 16.

The AAVrh79 vp1, vp2 and vp3 proteins contain subpopulations with amino acid modifications comprising at least two highly deamidated asparagines (N) in asparagine-glycine pairs in SEQ ID NO: 16 and optionally further comprising subpopulations comprising other deamidated amino acids, wherein the deamidation results in an amino acid change. High levels of deamidation at N-G pairs N57, N263, N385 and/or N514 are observed, relative to the number of SEQ ID NO: 16. Deamidation has been observed in other residues, as shown in the table below and in the examples. In certain embodiments, AAVrh79 may have other residues deamidated, e.g., typically at less than 10% and/or may have other modifications, including methylations (e.g, -R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g, at one or more of W248, W307, W307, M405, M437, M473, W480, W480, W505, M526, M544, M561, W621, M637, and/or W697). Optionally the W may oxidize to kynurenine.

TABLE 1
AAVrh79 Deamidation

| AAVrh79 | |
|---|---|
| Deamidation based | |
| on VP1 numbering | % Deamidation |
| N57 + Deamidation | 65-90, 70-95, |
| | 80-95, 75-100, |
| | 80-100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, |
| | 80-95, 75-100, |
| | 80-100, or 90-100 |
| ~N410 + Deamidation | 1-25, |
| N479 + Deamidation | 1-5, 1-3 |
| ~N514 + Deamidation | 65-90, 70-95, |
| | 80-95, 75-100, |
| | 80-100, or 90-100 |
| ~Q601 + Deamidation | 0-1 |
| N653 + Deamidation | 0-2 |

In certain embodiments, an AAVrh79 capsid is modified in one or more of the positions identified in the preceding table, in the ranges provided below, as determined using mass spectrometry with a trypsin enzyme. In certain embodiments, one or more of the following positions, or the glycine following the N is modified as described herein. Residue numbers are based on the AAVrh79 sequence provided herein. See, SEQ ID NO: 16.

In certain embodiments, the nucleic acid sequence encoding the AAVrh79 vp1 capsid protein is provided in SEQ ID NO: 15. In other embodiments, a nucleic acid sequence of 70% to 99.9% identity to SEQ ID NO: 15 may be selected to express the AAVrh79 capsid proteins. In certain other embodiments, the nucleic acid sequence is at least about 75% identical, at least 80% identical, at least 85%, at least 90%, at least 95%, at least 97% identical, at least 99% or at least 99.9% identical to SEQ ID NO: 15. However, other nucleic acid sequences which encode the amino acid sequence of SEQ ID NO: 16 may be selected for use in producing rAAV capsids. In certain embodiments, the nucleic acid sequence has the nucleic acid sequence of SEQ ID NO: 15 or a sequence at least 70% to 99% identical, at least 75%, at least 80%, at least 85%, at least 90%, at least 95%, at least 97%, at least 99%, identical to SEQ ID NO: 15 which encodes SEQ ID NO: 16. In certain embodiments, the nucleic acid sequence has the nucleic acid sequence of SEQ ID NO: 15 or a sequence at least 70% to 99.%, at least 75%, at least 80%, at least 85%, at least 90%, at least 95%, at least 97%, at least 99%, identical to about nt 412 to about nt 2214 of SEQ ID NO: 15 which encodes the vp2 capsid protein (about aa 138 to 738) of SEQ ID NO: 16. In certain embodiments, the nucleic acid sequence has the nucleic acid sequence of about nt 610 to about nt 2214 of SEQ ID NO: 15 or a sequence at least 70% to 99.%, at least 75%, at least 80%, at least 85%, at least 90%, at least 95%, at least 97%, at least 99%, identical to nt SEQ ID NO: 15 which encodes the vp3 capsid protein (about aa 204 to 738) of SEQ ID NO: 16.

In certain embodiment, an rAAV79 vector has an AAVrh79 capsid containing a vector genome comprising a nucleic acid molecule comprising AAV inverted terminal repeat sequences and a non-AAV nucleic acid sequence encoding a product operably linked to sequences which direct expression of the product. In certain embodiments, the AAVrh79 capsid is characterized by comprising a heterogeneous population of AAVrh79 vp1 proteins, AAVrh79 vp2 proteins, and AAVrh79 vp3 proteins selected from: vp1 proteins produced by expression from a nucleic acid sequence which encodes the predicted amino acid sequence of 1 to 738 of SEQ ID NO: 16, vp1 proteins produced from SEQ ID NO: 15, or vp1 proteins produced from a nucleic acid sequence at least 70% to 100% identical to SEQ ID NO: 15 which encodes the predicted amino acid sequence of 1 to 738 of SEQ ID NO: 16, a heterogeneous population of AAVrh79 vp2 proteins selected from: vp2 proteins produced by expression from a nucleic acid sequence which encodes the predicted amino acid sequence of at least about amino acids 138 to 738 of SEQ ID NO: 16, vp2 proteins produced from a sequence comprising at least nucleotides 412 to 2214 of SEQ ID NO: 15, or vp2 proteins produced from a nucleic acid sequence at least 70% to 100% identical to at least nucleotides 412 to 2214 of SEQ ID NO: 15 which encodes the predicted amino acid sequence of at least about amino acids 138 to 738 of SEQ ID NO: 16, and a heterogeneous population of AAVrh79 vp3 proteins selected from: vp3 proteins produced by expression from a nucleic acid sequence which encodes the predicted amino acid sequence of at least about amino acids 204 to 738 of SEQ ID NO: 16, vp3 proteins produced from a sequence comprising at least nucleotides 610 to 2214 of SEQ ID NO: 15, or vp3 proteins produced from a nucleic acid sequence at least 70% identical to at least nucleotides 610 to 2214 of SEQ ID NO: 15 which encodes the predicted amino acid sequence of at least about amino acids 204 to 738 of SEQ ID NO: 16. In certain embodiments, the rAAVrh79 capsid is characterized by a heterogeneous population of AAVrh79 vp1 proteins, AAVrh79 vp2 and AAVrh79 vp3 proteins which are the product of a nucleic acid sequence encoding the amino acid sequence of SEQ ID NO: 16, a heterogeneous population of vp2 proteins which are the product of a nucleic acid sequence encoding the amino acid sequence of at least about amino acids 138 to 738 of SEQ ID NO: 16, and a heterogeneous population of vp3 proteins which are the product of a nucleic acid sequence encoding at least amino acids 204 to 738 of SEQ ID NO: 16. In certain embodiments, the AAVrh79 capsid is characterized by AAVrh79 vp1proteins, vp2 proteins and vp3 proteins which comprise heterogenous populations relative to amino acids 1 to 738 (vp1), 138 to 738 (vp2), and 204 to 738 (vp3), respectively, of SEQ ID NO: 16, wherein: the heterogenous population of AAVrh79 vp1, AAVrh79 vp2 and AAVrh79 vp3 proteins contain subpopulations with amino acid modifications comprising at least 50% to 100% two highly deamidated asparagines (N) in asparagine-glycine pairs in at least two positions relative to SEQ ID NO: 16 and optionally further comprising subpopulations comprising other deamidated amino acids, wherein the deamidation results in an amino acid change. In certain embodiment, the highly deamidated positions are N57, N263, N385, and N514, based on SEQ ID NO: 16, and as measured using mass spectrometry. In certain embodiment, the AAVrh79 capsid proteins are each individually deamidated at 60% to about 100% at position N57, at 60% to about 100% at position N263, at 60% to about 100% at position N385, and at 60% to about 100% at position N514, based on SEQ ID NO: 16, and as measured using mass spectrometry. Other suitable techniques for measuring deamidation or other post-translational modifications may be selected.

In certain embodiments, the rAAV79 capsid comprises AAVrh79 VP1 proteins having about 80 to 85% deamidation at position N57 of SEQ ID NO: 16; about 82% to about 88% deamidation at position N263 of SEQ ID NO: 16; about 90% to about 96% deamidation at position N385 of SEQ ID NO: 3; and/or about 85% to about 90% deamidation at position N514 of SEQ ID NO: 16, optionally with further post-translational modifications at other positions, as determined using mass spectrometry. Optionally, there is deamidation at position N94, N254, N305, N410, N479, Q601, N653; generally deamidation in found in these positions at less than 10% of the population of VP1, VP2 and VP3 proteins of AAVrh79, less than 5%, less than 3%, or less than 2%. Optionally, phosphorylation is observed at position S149, based on the residues of SEQ ID NO: 16; in certain embodiments, no more than 0% of the capsid proteins have phosphorylation at this position. In certain embodiments, oxidation is observed at positions W248, W307, M437, M473, M480, W505, M637, and/or W697; in certain embodiments, less than 10% of the capsid proteins are oxidated at any one of these positions. Post-translational modifications may be determined using mass spectrometry or another suitable technique.

The invention also encompasses nucleic acid sequences encoding mutant AAVrh79, in which one or more residues has been altered in order to decrease deamidation, or other modifications which are identified herein. Such nucleic acid sequences can be used in production of mutant rAAVrh79 capsids.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, expression cassette containing the transgene or coding sequence(s) operably linked to regulatory sequences directing expression thereof, and an AAV 3′ ITR. The ITRs are the genetic elements responsible for the replication and packaging of the genome during vector production and are the only viral cis elements required to generate rAAV. In a preferred embodiment, the ITR sequences from AAV2, or the deleted version thereof (ΔITR), which may be used for convenience. In certain embodiments, the ITRs are those shown in nucleotides 1 to 130 and 3052 to 3181 of SEQ ID NO: 2, and nucleotides 1 to 130 and 3345 to 3474 of SEQ ID NO: 6.

A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length AAV 5′ and 3′ ITRs are used. In other embodiments, a full-length or engineered ITR may be selected. ITRs from AAV2, a different source AAV than the capsid, or other than full-length ITRs may be selected. The ITRs are from the same AAV source as the AAV which provides the rep function during production or a transcomplementing AAV.

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin Biotechnol. 99: 119-145; Buning et al., 2008, “Recent developments in adeno-associated virus vector technology,” J Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Publication No. WO 2017/160360, which is incorporated by reference herein. Purification methods for AAV8, International Patent Publication No. WO 2017/100676, and rh10, International Patent Publication No. WO 2017/100704, and for AAV1, International Patent Publication No. WO 2017/100674 are all incorporated by reference herein.

To calculate empty and full particle content, VP3 band volumes for a selected sample (e.g., in examples herein an iodixanol gradient-purified preparation where #of GC=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14. The ddPCR method directly measures the concentration of encapsidated vector genomes. The sample is treated with DNase I to digest any non-encapsidated DNA present in the sample followed by treatment with Proteinase K to disrupt the capsid. The sample is then diluted to fit the assay range. The sample is mixed with ddPCR Supermix, and detection is accomplished using sequence-specific primers targeting the Meganuclease specific to the PCSK9 gene (ARCUS) in combination with a fluorescently-tagged probe hybridizing to this same region. Twenty microliters of ddPCR reaction mixture is processed in the Bio-Rad droplet generator, and the ddPCR reaction mixture is partitioned into >10,000 droplets. After droplet generation, the ddPCR reaction mixture undergoes PCR amplification, and the amplified ddPCR reaction mixture is read using a Bio-Rad Droplet Reader.

The infectious unit (IU) assay may be used to determine the productive uptake and replication of rAAV vector in RC32 cells (rep2 expressing HeLa cells). A 96-well endpoint format has been employed similar to that previously published. Briefly, RC32 cells will be co-infected by serial dilutions of rAAV BDS and a uniform dilution of Ad5 with 12 replicates at each dilution of rAAV. Seventy-two hours after infection, the cells will be lysed, and qPCR will be performed to detect rAAV vector amplification over input. An endpoint dilution 50% tissue culture infectious dose (TCID₅₀) calculation (Spearman-Karber) will be performed to determine a infectious titer expressed as IU/mL. Since “infectivity” values are dependent on each particle's contact with cells, receptor binding, internalization, transport to the nucleus, and genome replication, they are influenced by assay geometry and the presence of appropriate receptors and post-binding pathways in the cell line used. Receptors and post-binding pathways are not usually maintained in immortalized cell lines, and thus infectivity assay titers are not an absolute measure of the number of “infectious” particles present. However, the ratio of encapsidated GC to “infectious units” (described as GC/IU ratio) can be used as a measure of product consistency from lot to lot.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1)] which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Dual Vector System

In another aspect, a dual vector system for treating a genetic disorder is provided. The system includes (a) a gene editing component that includes a nucleic acid sequence encoding a meganuclease that targets PCSK9 and regulatory sequences that direct expression of the nuclease in a target cell comprising a PCSK9 gene; and (b) a donor vector comprising a nucleic acid sequence encoding OTC for expression from the PCSK9 locus, and wherein the system further comprises sequences that direct the nuclease to specifically targets the native PCSK9 gene locus. The components of the dual vector are as those described herein.

In one embodiment, the expression cassette of the gene editing vector includes the sequence of nucleotides 211 to 2964 of SEQ ID NO: 2 or a sequence sharing at least 80%, at least 85%, at least 90%, at least 95%, at least 96%, at least 97%, at least 98%, at least 99%, or at least 99.9% identity to the sequence of nucleotides 211 to 2964 of SEQ ID NO: 2.

In one embodiment, the expression cassette of the donor vector includes the sequence of nucleotides 178 to 3281 of SEQ ID NO: 6 or a sequence sharing at least 80%, at least 85%, at least 90%, at least 95%, at least 96%, at least 97%, at least 98%, at least 99%, or at least 99.9% identity to the sequence of nucleotides 178 to 3281 of SEQ ID NO: 6.

While the system can be effective if the ratio of gene editing vector to template vector is about 1 to about 1, it is desirable for the donor template vector to be present in excess of the gene editing vector. In one embodiment, the ratio of editing vector (a) to donor vector (b) is about 1:3 to about 1:100, or about 1:10. In certain embodiments, the ratio of editing vector (a) to donor vector (b) is about 1:3. In certain embodiments, the ratio of editing vector (a) to donor vector (b) is about 1:2. In certain embodiments, the ratio of editing vector (a) to donor vector (b) is about 1:2.5. In certain embodiments, the ratio of editing vector (a) to donor vector (b) is about 1:3.5. In certain embodiments, the ratio of editing vector (a) to donor vector (b) is about 1:4. In certain embodiments, the ratio of editing vector (a) to donor vector (b) is about 1:4.5. In certain embodiments, the ratio of editing vector (a) to donor vector (b) is about 1:5.

In one embodiment, the dual vector system includes a gene editing AAV comprising an AAV capsid and a first vector genome comprising a 5′ ITR, a sequence encoding a meganuclease that targets PCSK9 under control of regulatory sequences that direct expression of the meganuclease in a target cell comprising a PCSK9 gene, and a 3′ ITR; and (b) a donor AAV vector comprising an AAV capsid and a second vector genome comprising: a 5′ITR, a 5′ homology directed recombination (HDR) arm, an OTC transgene and regulatory sequences that direct expression of the transgene in the target cell, a 3′ HDR arm, and a 3′ ITR.

### Pharmaceutical Compositions

In another aspect, a pharmaceutical composition is provided which contains a first rAAV stock comprising rAAV gene editing vectors comprising an expression cassette comprising a nucleic acid sequence encoding a meganuclease that targets PCSK9 (e.g., the protein sequence of SEQ ID NO: 3) and regulatory sequences that direct expression of the nuclease in a target cell comprising a PCSK9 gene; and a second rAAV stock comprising rAAV donor vectors comprising a transgene cassette comprising a nucleic acid sequence encoding an OTC transgene (e.g., coding sequence of SEQ ID NO: 4) and regulatory sequences that direct expression of the transgene in the target cell. The pharmaceutical composition contains an optional carrier, excipient, and/or preservative. In some embodiments, the donor vector further includes homology-directed recombination (HDR) arms 5′ and 3′ to the transgene cassette. In one embodiment, the AAV capsid for the donor vector, gene editing vector, or both, is an AAVrh79 capsid.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney,), oral, inhalation, intranasal, intrathecal, intratracheal, intraarterial, intraocular, intravenous, intramuscular, subcutaneous, intradermal, and other parental routes of administration. In certain embodiments, the route of administration is IV. Routes of administration may be combined, if desired.

Dosages of the viral vector depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method.

Any suitable route of administration may be selected. Accordingly, pharmaceutical compositions may be formulated for any appropriate route of administration, for example, in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

In one aspect, provided herein is a pharmaceutical composition comprising at least parvovirus vector comprising at least one gene editing vector and at least one donor vector as described herein in a formulation buffer. In certain embodiments, the pharmaceutical composition comprises a combination of different vector populations. In one embodiment, provided is a pharmaceutical composition comprising a single rAAV population described herein in a formulation buffer. The methods provided herein provide for co-administration of two separate vector-containing suspensions.

### Methods

The compositions provided herein are useful for treatment of omithine transcarbamylase deficiency. Provided herein is a method of treating a disorder in a human by co-administering the dual vector system as described herein. In certain embodiments, provided herein is a composition comprising non-replicating recombinant adeno-associated virus serotype rh79 vectors: AAVrh79.TBG.M2PCSK9.WPRE.bGH and AAVrh79.hHDR.TBG.hOTCco.bGH for treatment of omithine transcarbamylase deficiency.

In one embodiment, a method of treating OTC deficiency in a subject is provided. The method includes co-administering to the subject having OTC deficiency a gene editing AAV vector comprising a sequence encoding a nuclease that targets PCSK9 and regulatory sequences that direct expression of the nuclease in a target cell comprising a PCSK9 gene; and a donor AAV vector comprising a transgene and regulatory sequences that direct expression of the OTC transgene in the target cell. In one embodiment, the subject is a neonate.

In certain embodiments, the gene editing AAV vector and the donor vector of are delivered essentially simultaneously via the same route. In other embodiments, the gene editing vector is delivered first. In other embodiments, the donor vector is delivered first.

In one embodiment, the dosage of an rAAV is about 1×10⁹ GC to about 1×10¹⁵ genome copies (GC) per dose (to treat an average subject of 70 kg in body weight), and preferably 1.0×10¹² GC to 2.0×10¹⁵ GC for a human patient. In another embodiment, the dose is less than about 1×10¹⁴ GC/kg body weight of the subject. In certain embodiments, the dose administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10⁹ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×10¹⁰ GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×10¹¹ GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×10¹³ GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg body weight or the subject.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, IV administration is used.

The system described herein may be therapeutically useful if a sufficient amount of functional enzyme or protein is generated to improve the patient's condition. In certain embodiments, gene expression levels as low as 5% of the level of a healthy patients will provide sufficient therapeutic effect for the patient. In other embodiments, gene expression levels are at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the normal range (levels) observed in humans (or other veterinary subject). For example, by “functional enzyme”, is meant a gene which encodes the wild-type enzyme (e.g., OTCase) which provides at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%6, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about the same, or greater than 100% of the biological activity level of the wild-type enzyme, or a natural variant or polymorph thereof which is not associated with disease. Similarly, patients having no detectable amounts of enzyme may be rescued by delivering enzyme function to less than 100% activity levels, and may optionally be subject to further treatment subsequently. In certain embodiments, where gene function is being delivered by the donor template, patients may express higher levels than found in “normal”, healthy subjects. As described herein, the therapy described herein may be used in conjunction with other treatments, i.e., the standard of care for the subject's (patient's) diagnosis.

In one embodiment, the method further comprises administering an immunosuppressive co-therapy to the subject. Such immunosuppressive co-therapy may be started prior to delivery of an rAAV or a composition as disclosed, e.g., if undesirably high neutralizing antibody levels to the AAV capsid are detected. In certain embodiments, co-therapy may also be started prior to delivery of the rAAV as a precautionary measure. In certain embodiments, immunosuppressive co-therapy is started following delivery of the rAAV, e.g., if an undesirable immune response is observed following treatment.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include prednisolone, a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the rAAV administration, or 0, 1, 2, 3, 7, or more days post the rAAV administration. Such therapy may involve a single drug (e.g., prednisolone) or co-administration of two or more drugs, the (e.g., prednisolone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), two weeks, three weeks, about 60 days, or longer, as needed. In certain embodiments, a tacrolimus-free regimen is selected.

In another embodiment, the method includes co-treatment with a standard OTC therapy. Treatment of OTC deficiency is largely focused on dietary management of blood ammonia levels to avoid hyperammonemia or to remove excess ammonia from the blood during hyperammonemic episodes (NORD, 2021). Individuals with OTC deficiency follow dietary restrictions limiting their protein intake to control blood ammonia levels. Dietary restrictions must be carefully balanced in infants, who need to consume enough protein to ensure proper growth while avoiding excess protein intake that could trigger a hyperammonemic episode (Berry and Steiner, 2001). As such, infants are placed on high calorie, low protein diets supplemented by essential amino acids. In hyperammonemic episodes, all protein may be removed from a patient's diet for 24 hours (NORD, 2021).

There are several medications designed to stimulate the removal of nitrogen from the blood stream. Sodium phenylbutyrate (Buphenyl) is approved by the United States Food and Drug Administration (FDA) for the treatment of chronic hyperammonemia in patients with OTC deficiency. Once metabolized, Buphenyl is converted to phenylacetate, which conjugates with glutamine to form phenylacetylglutamine, which is excreted by the kidney, providing an alternative pathway for nitrogen excretion. Glycerol phenylbutyrate (Ravicti) is also FDA-approved for the treatment of chronic hyperammonemia in patients with urea cycle disorders. Like Buphenyl, Ravicti is converted to phenylacetate and follows the same mechanism for excreting nitrogen (Lichter-Konecki et al., 1993; Gordon, 2003; Magellan, 2021). Finally, Ammonul (sodium phenylacetate and sodium benzoate), is approved by the FDA as an adjunctive therapy for the treatment of acute hyperammonemia in patients with urea cycle disorders. The sodium phenylacetate component of Ammonul follows the same mechanism for nitrogen excretion as the phenylacetate metabolite created by Buphenyl and Ravicti. The sodium benzoate component of Ammonul conjugates with glycine to form hippuric acid, which is excreted by the kidneys and removing nitrogen through this process. Sodium benzoate is also given as an oral preparation for long-term maintenance of OTC deficiency and is often preferred over Buphenyl and Ravicti since it thought to have fewer side effects (Lichter-Konecki et al., 1993).

Many people with OTC deficiency are treated with arginine or citrulline, which are needed to ensure protein synthesis proceeds at a normal rate. Citrulline is often chosen for chronic treatment over arginine because it incorporates aspartate into the urea cycle, meaning it contributes one additional nitrogen molecule into this pathway (Lichter-Konecki et al., 1993; Magellan, 2021).

In hyperammonemic episodes that progress to vomiting and lethargy, hospitalization may be required and individuals may be treated with an IV infusion containing arginine and Ammonul. If these treatments are unsuccessful, hemofiltration or hemodialysis may be required to rapidly lower blood ammonia levels (Lichter-Konecki et al., 1993).

Liver transplantation is the most effective strategy for preventing hyperammonemic crises and neurodevelopmental deterioration in patients with severe, neonatal onset OTC deficiency. In patients with mild or well-managed OTC deficiency, stressors can cause life-threatening hyperammonemic episodes at any age. The fear of such episodes, in addition to the diminished quality of life these patients face due to dietary restrictions and chronic treatment with nitrogen scavengers that can be accompanied by significant side effects motivate many patients to seek liver transplants even if their condition is well-managed (Lichter-Konecki et al., 1993).

In one aspect, a method is provided for treating a patient having ornithine transcarbamylase (OTC) deficiency, using a nuclease expression cassette comprising a meganuclease which recognizes a site within the human PCSK9 gene, under the control of a TBG promoter as described herein. The method further includes administration of an expression cassette carrying the OTC transgene of SEQ ID NO: 4, or a sequence sharing at least 90% identity therewith, as described herein.

A variety of assays exist for measuring OTC expression and activity levels in vitro. See, e.g., X Ye, et al, 1996 Prolonged metabolic correction in adult ornithine transcarbamylase-deficient mice with adenoviral vectors. J Biol Chem 271:3639-3646) or in vivo. For example, OTC enzyme activity can be measured using a liquid chromatography mass spectrometry stable isotope dilution method to detect the formation of citrulline normalized to [1,2,3,4,5-13C5] citrulline (98% 13C). The method is adapted from a previously developed assay for detection of N-acetylglutamate synthase activity [Morizono H, et al, Mammalian N-acetylglutamate synthase. Mol Genet Metab. 2004; 81(Suppl 1):54-11.]. Slivers of fresh frozen liver are weighed and briefly homogenized in buffer containing 10 mM HEPES, 0.5% Triton X-100, 2.0 mM EDTA and 0.5 mM DTT. Volume of homogenization buffer is adjusted to obtain 50 mg/ml tissue. Enzyme activity is measured using 250 μg liver tissue in 50 mM Tris-acetate, 4 mM ornithine, 5 mM carbamyl phosphate, pH 8.3. Enzyme activity is initiated with the addition of freshly prepared 50 mM carbamyl phosphate dissolved in 50 mM Tris-acetate pH 8.3, allowed to proceed for 5 minutes at 25° C. and quenched by addition of an equal volume of 5 mM13C5-citrulline in 30% TCA. Debris is separated by 5 minutes of microcentrifugation, and the supernatants are transferred to vials for mass spectroscopy. Ten μL of sample are injected into an Agilent 1100 series LC-MS under isocratic conditions with a mobile phase of 93% solvent A (1 ml trifluoroacetic acid in 1 L water):7% solvent B (1 ml trifluoroacetic acid in 1 L of 1:9 water/acetonitrile). Peaks corresponding to citrulline [176.1 mass:charge ratio (m/z)] and 13C5-citrulline (181.1 m/z) are quantitated, and their ratios are compared to ratios obtained for a standard curve of citrulline run with each assay. Samples are normalized to either total liver tissue or to protein concentration determined using a Bio-Rad protein assay kit (Bio-Rad, Hercules, CA). Other assays, which do not require liver biopsy, may also be used. One such assay is a plasma amino acid assays in which the ratio of glutamine and citrulline is assessed and if glutamine is high (>800 microliters/liter) and citrulline low (e.g., single digits), a urea cycle defect is suspected. Plasma ammonia levels can be measured and a concentration of about 100 micromoles per liter is indicative of OTCD. Blood gases can be assessed if a patient is hyperventilating; respiratory alkalosis is frequent in OTCD. Orotic acid in urine, e.g., greater than about 20 micromoles per millimole creatine is indicative of OTCD, as is elevated urinary orotate after allopurinol challenge test. Diagnostic criteria for OTCD have been set forth in Tuchman et al, 2008, Urea Cycle Disorders Consortium (UCDC) of the Rare Disease Clinical Research Network (RDCRN). Tuchman M, et al., Consortium of the Rare Diseases Clinical Research Network. Cross-sectional multicenter study of patients with urea cycle disorders in the United States. Mol Genet Metab. 2008; 94:397-402, which is incorporated by reference herein. See, also, http://www.ncbi.nlm.nih.gov/books/NBK154378/, which provides a discussion of the present standard of care for OTCD.

In certain embodiments, a nuclease expression cassette, viral vector (e.g., rAAV), or any of the same in a pharmaceutical composition, as described herein is administrable for gene editing in a patient. In certain embodiments, the method is useful for non-embryonic gene editing. In certain embodiments, the patient is an infant (e.g., birth to about 4 months). In certain embodiments, the patient is older than an infant, e.g., 12 months or older.

As used herein, “a,” “an,” or “the” can mean one or more than one. For example, “a” cell can mean a single cell or a multiplicity of cells.

As used herein, the term “specificity” means the ability of a nuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

The (gene editing and donor) expression cassettes described herein, may be engineered into any suitable genetic element for delivery to a target cell, such as a vector. A “vector” as used herein is a biological or chemical moiety comprising a nucleic acid sequence which can be introduced into an appropriate host cell for replication or expression of said nucleic acid sequence.

“Plasmid” or “plasmid vector” generally is designated herein by a lowercase p preceded and/or followed by a vector name. Plasmids, other cloning and expression vectors, properties thereof, and constructing/manipulating methods thereof that can be used in accordance with the present invention are readily apparent to those of skill in the art.

As used herein, the term “operably linked” refers to both expression control sequences that are contiguous with the gene of interest and expression control sequences that act in trans or at a distance to control the gene of interest.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same expression cassette or host cell, but which is present in a non-natural state, e.g. a different copy number, or under the control of different regulatory elements.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene.

As used herein, the term “host cell” may refer to the packaging cell line in which a vector (e.g., a recombinant AAV) is produced from a production plasmid. In the alternative, the term “host cell” may refer to any target cell in which expression of the transgene is desired. Thus, a “host cell,” refers to a prokaryotic or eukaryotic cell that contains a exogenous or heterologous nucleic acid sequence that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. Still in other embodiment, the term “host cell” is intended to reference the target cells of the subject being treated in vivo for the diseases or conditions as described herein. In certain embodiments, the term “host cell” is a liver cell or hepatocyte.

A “subject” is a mammal, e.g., a human, mouse, rat, guinea pig, dog, cat, horse, cow, pig, or non-human primate, such as a monkey, chimpanzee, baboon or gorilla. A patient refers to a human. A veterinary subject refers to a non-human mammal. In certain embodiments, the subject does not have a defect in their PCSK9 gene.

In certain embodiments, the subject has, or is at risk of developing, OTC deficiency. In certain embodiments, the subject has a documented genetic confirmation of an OTCD mutation. In certain embodiments, the subject has, or has had previously, Hyperammonemic Crisis (HAC). In certain embodiments, the subject is currently being treated for OTC deficiency, e.g., with at least one nitrogen scavenger therapy and/or a protein restricted diet.

In certain embodiments, the subject is male. In certain embodiments, it is desirable to treat the subject within hours of birth, e.g., at least 12 hours of age, 24 hours of age, 36 hours of age, or 48 hours of age. In other embodiments, it is desirable to treat the subject within days of birth, e.g., at 1, 2, 3, 4, 5, 6, 7, 8, 9, 10, 11, 12, 13 or 14 days from birth. In other embodiments, it is desirable to treat the subject within weeks of birth, e.g., at 1, 2, 3, 4, 5, 6, 7, 8, 9, 10, 11, 12, 13, 14, 15, or 16 weeks from birth. In other embodiments, it is desirable to treat the subject within months of birth, e.g., at 1, 2, 3, 4, 5, 6, 7, 8, 9, 10, 11, 12, 13, 14, 15, 16, 17, 18, 19, 20, 21, 22, 23 or 24 months from birth. In certain embodiments, the subject is treated at 24 hours to 4 months of age. In some embodiments, the subject is a male infant treated at 24 hours to 4 months of age with documented genetic confirmation of an OTCD mutation consistent with neonatal onset OTC deficiency with or without current or past Hyperammonemic Crisis (HAC) and currently being treated with at least one nitrogen scavenger therapy and a protein restricted diet. The proposed study population includes subjects with high unmet need and in whom it is possible to observe stabilization or improvement in frequency and severity of recurrent hyperammonemic events owing to decreased morbidity and mortality, and increased survival, while possibly delaying the need for hepatic transplant within a 52-week protocol timeframe.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the term “about” refers to a variant of 10% from the reference integer and values therebetween. For example, “about” 40 base pairs, includes ±4 (i.e., 36-44, which includes the integers 36, 37, 38, 39, 40, 41, 42, 43, 44). For other values, particularly when reference is to a percentage (e.g., 90% identity, about 10% variance, or about 36% mismatches), the term “about” is inclusive of all values within the range including both the integer and fractions.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

We describe a genome editing approach for the treatment of ornithine transcarbamylase deficiency (OTCD) which can cause lethal episodes of hyperammonemia in infancy. The goal of genome editing is for the therapeutic effect to be durable and achieved in all OTCD patients independent of their mutation. We accomplish this by treating surviving newborns with two AAV vectors: one to deliver a nuclease to create a double stranded break in a safe-harbor stie and the second to deliver an OTC minigene for knock-in into this site. Our assumption is that dividing hepatocytes of the newborn liver will be conducive to efficient knock-in of the OTC gene and will eliminate, through dilution, the non-integrated input vector genomes. We decided to use the PCSK9 gene as a safe-harbor site and a meganuclease, called ARCUS to target it, based on our previous work in adult macaques which showed safe, efficient and stable reductions of PCSK9 following AAV delivery ARCUS. Our initial studies of genome editing for OTCD were performed in an OTC deficient mouse rendered susceptible to the PCSK9 ARCUS nuclease through germ line modification of exon 7 of the endogenous PCSK9 gene. Injection of the two vectors into newborn mice resulted in efficient knock-in of the human OTC minigene and protection to lethal hyperammonemia when challenged with a high protein diet. In preparation for clinical studies, we evaluated key safety and efficacy parameters in newborn and infant macaques. A total of 24 animals were treated with AAV vectors with analyses to include examination of liver biopsies at 3 and 12 months. In these studies we evaluated the impact of the following parameters on editing efficiency and toxicity: transgene (human factor IX and human OTC), promoters driving ARCUS, Clade E capsids, length of donor flanking the transgene and age of the macaque at time of dosing. We found the injection of AAV vectors was quite safe with no evidence of transaminase elevations or liver histopathology in any ARCUS treated animals. The key measure of efficacy in the primate model is transduction efficiency measured by in situ hybridization and immunostaining to detect cells expressing the human OTC mRNA and protein, respectively. The highest and most consistent results were obtained with vectors using a novel clade E capsid driving ARCUS with a TBG promoter in the first vector and using 500 bp flanking homology arms on the donor vector. With this combination we achieved 10.0±6.4% (N=6) transduction which is higher than the threshold we believe can provide substantial benefit to patients which is ˜5% OTC expressing cells. Preliminary data suggests that the level of editing is stable over one year and that efficient targeted insertion can be achieved when injected into macaques up to 3 months of age. Molecular analyses of the PCSK9 target locus suggested the vast majority of the knocked-in of vector genome were through non-homologous end joining (NHEJ) rather than homology-directed repair (HDR). In summary, the substantial unmet need of the neonatal form of OTCD warrants consideration of experimental therapies such as genome editing such as that described in this report.

### Example 1—Materials and Methods

AAV vectors were constructed according to previously established procedures and manufacturer's instructions. The AAVhu37 or AAVrh79 capsids were used for the experiments as described herein, where indicated.

All animal procedures were performed in accordance with protocols approved by the Institutional Animal Care and Use Committee of the University of Pennsylvania.

### Example 2—ARCUS2-Mediated hOTC Gene Targeting in Newborn NHPs

Newborn (1-16 days old) or infant (3-4 months old) rhesus macaques were used in non-GLP-compliant POC pharmacology studies. The ARCUS meganuclease targets a 22-bp sequence present in the human and rhesus macaque PCSK9 gene. Thus, rhesus macaques can be used to evaluate on-target editing (pharmacology) and safety/toxicology. Furthermore, newborn and infant rhesus macaques have similar anatomical and physiological features as human infants and will allow for the use of the intended clinical ROA (IV). It is anticipated that the similarity in anatomy and ROA will result in representative vector distribution and transduction profiles, which will enable more accurate assessment of the pharmacology and toxicity of the test article, including on- and off-target editing, and clinical pathology, which is not possible in newborn mice.

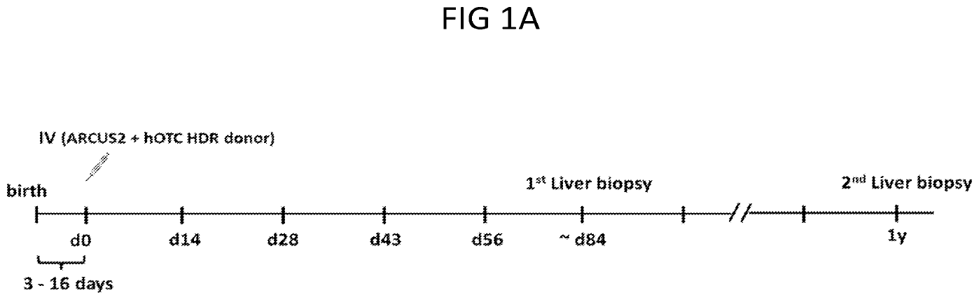

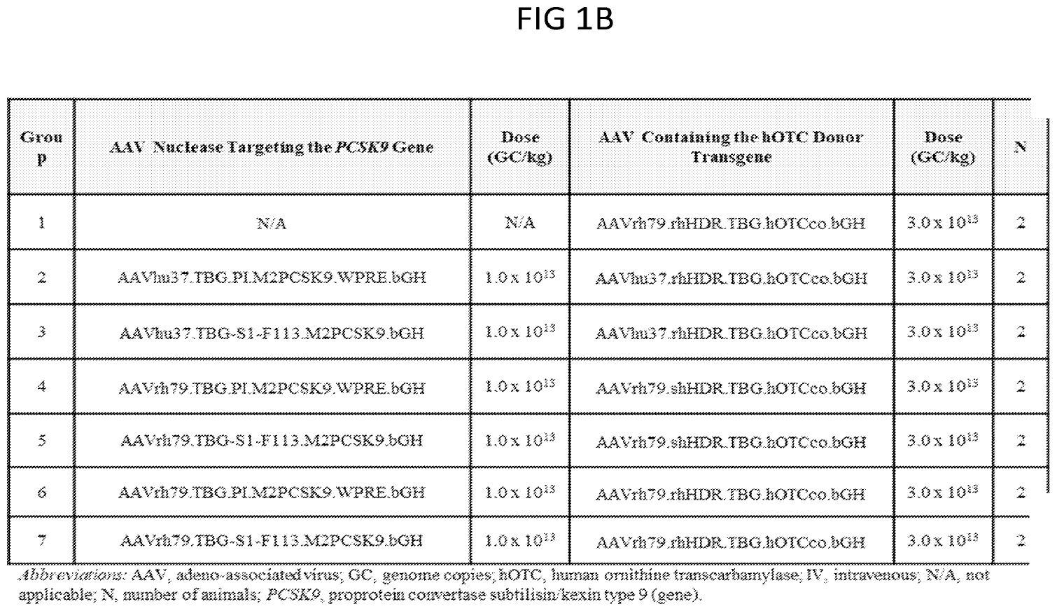

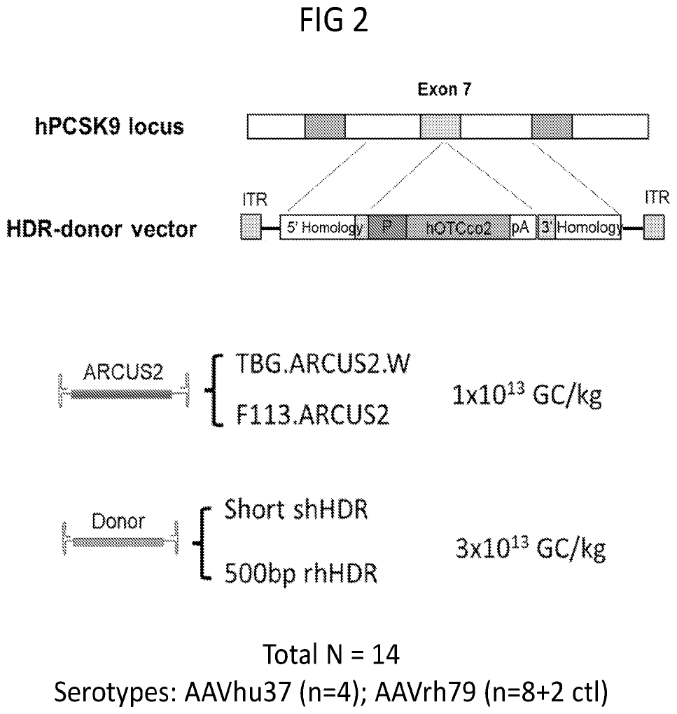

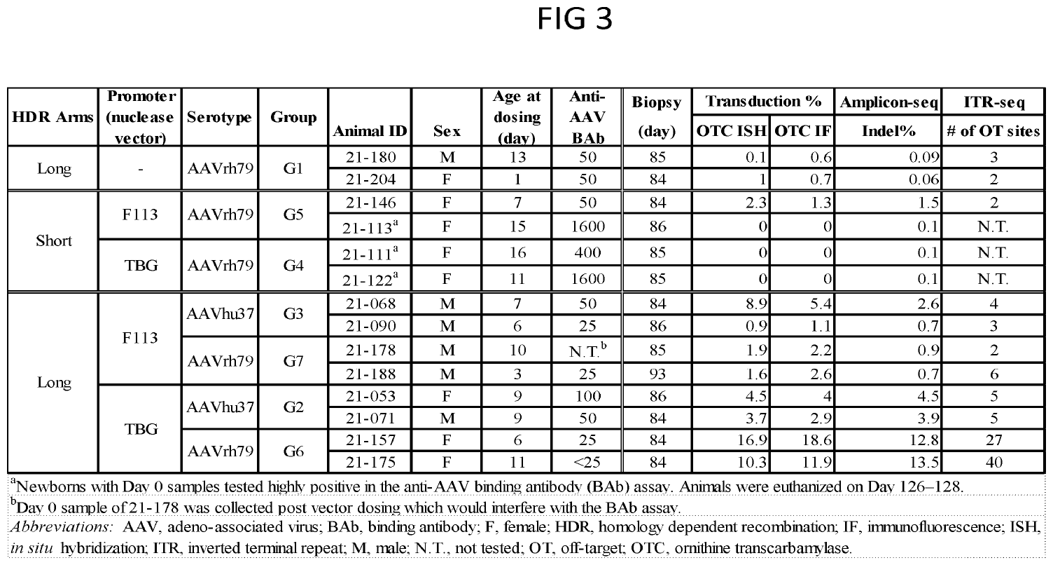

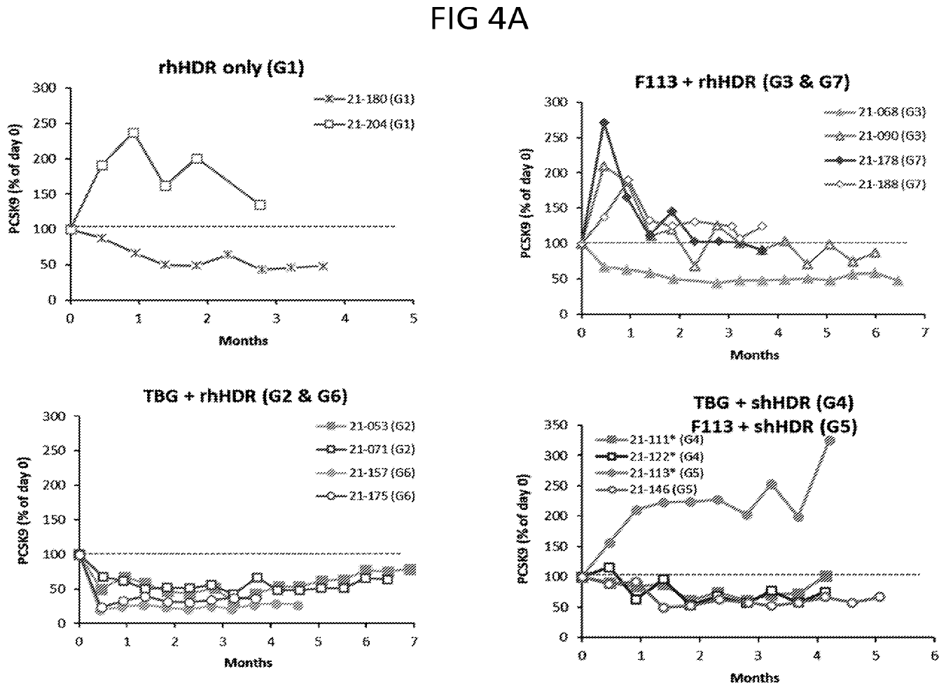

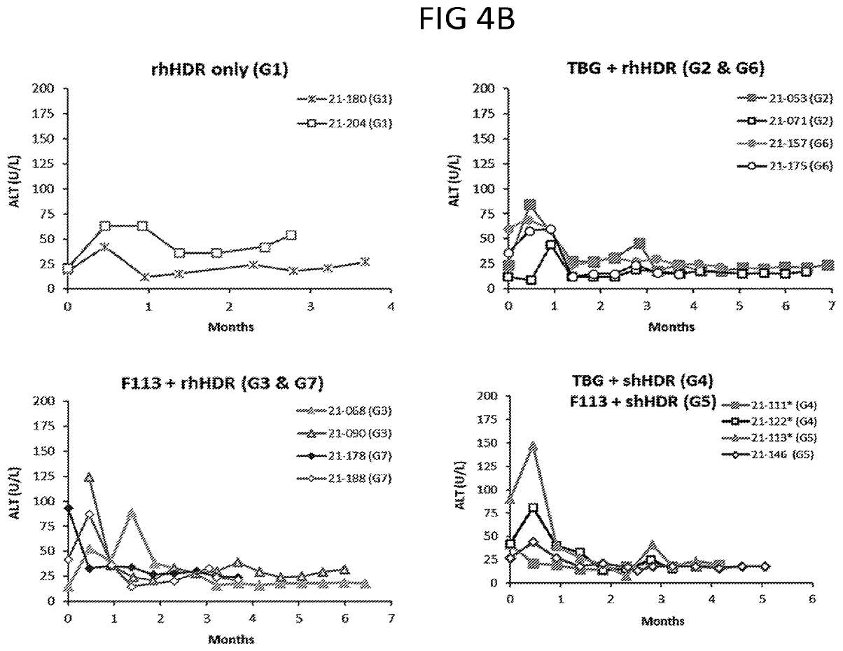

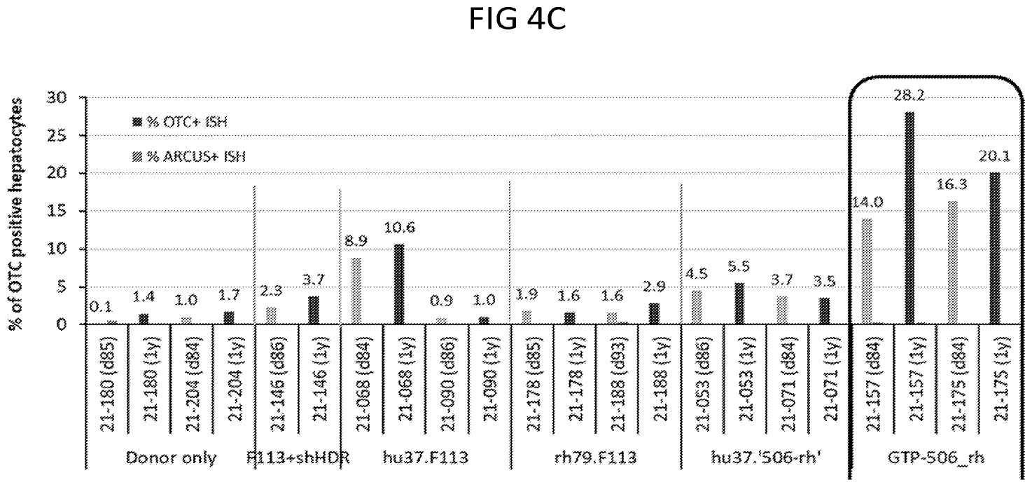

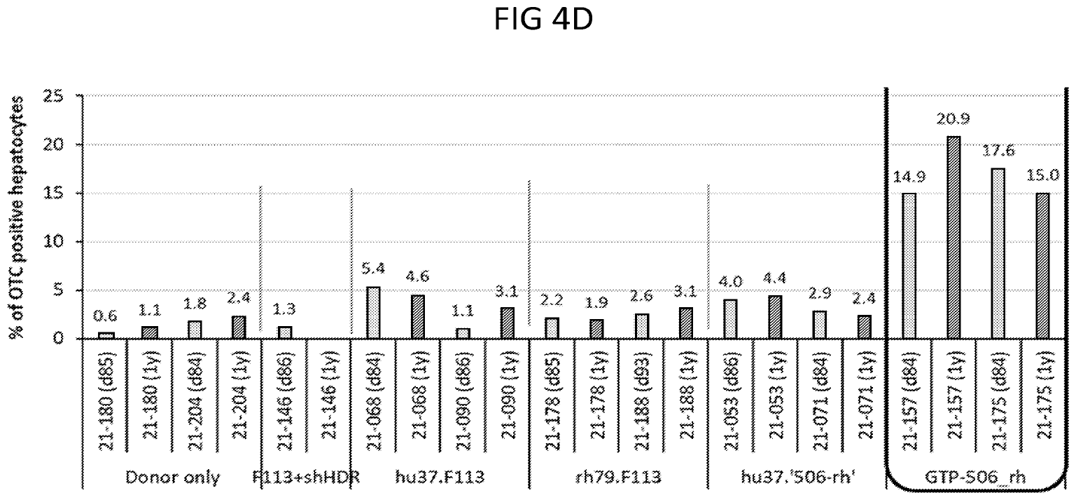

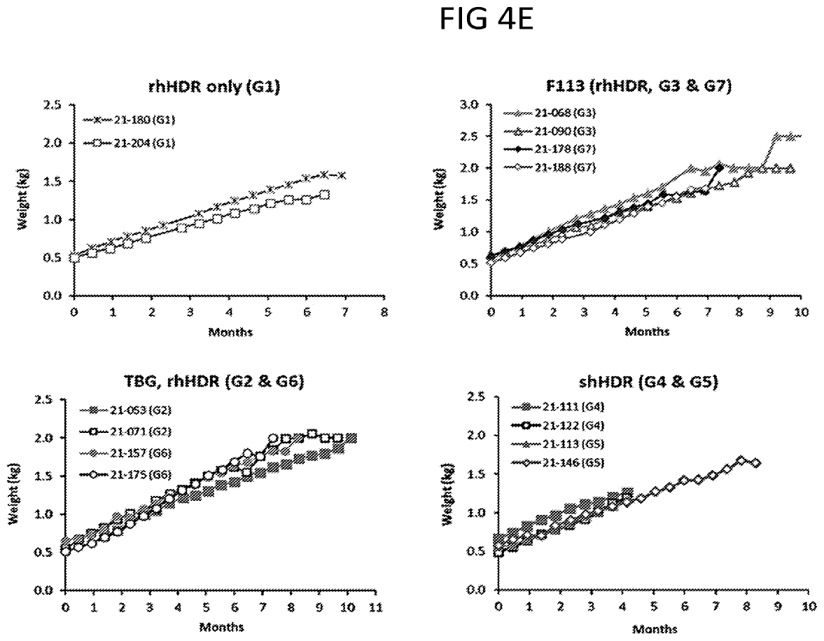

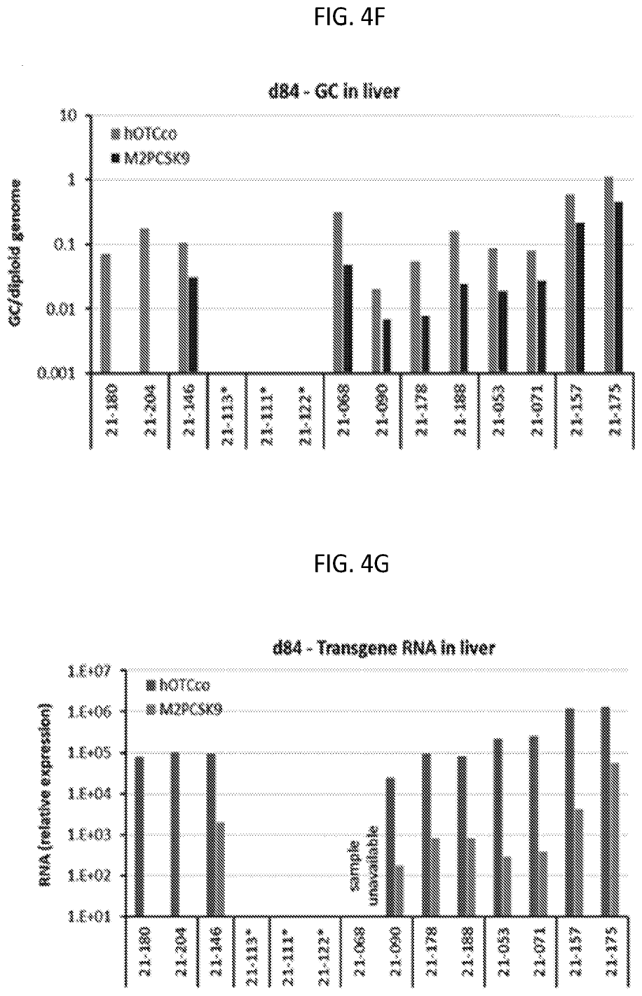

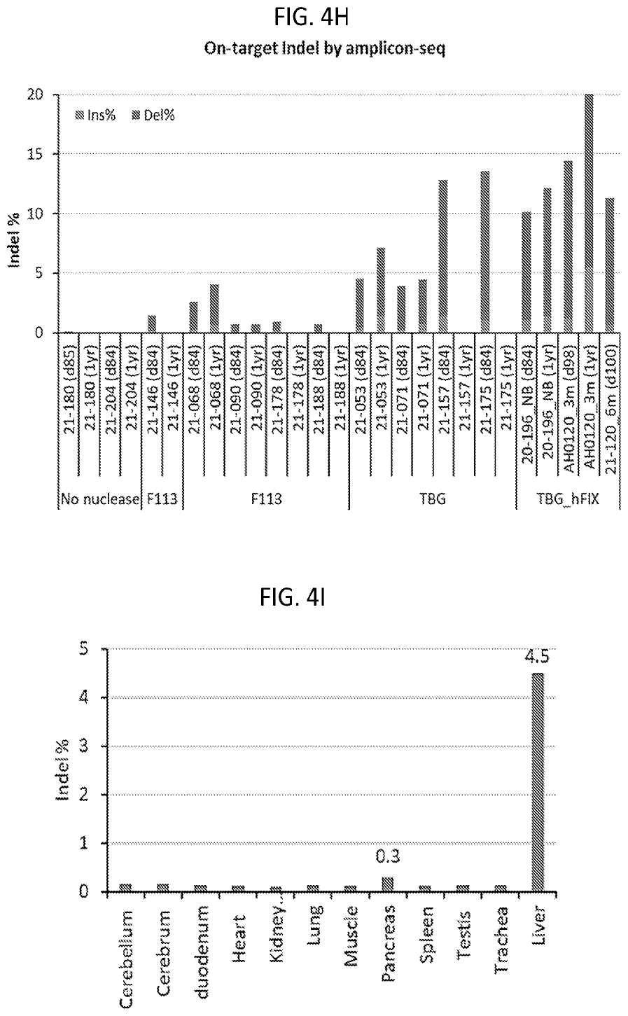

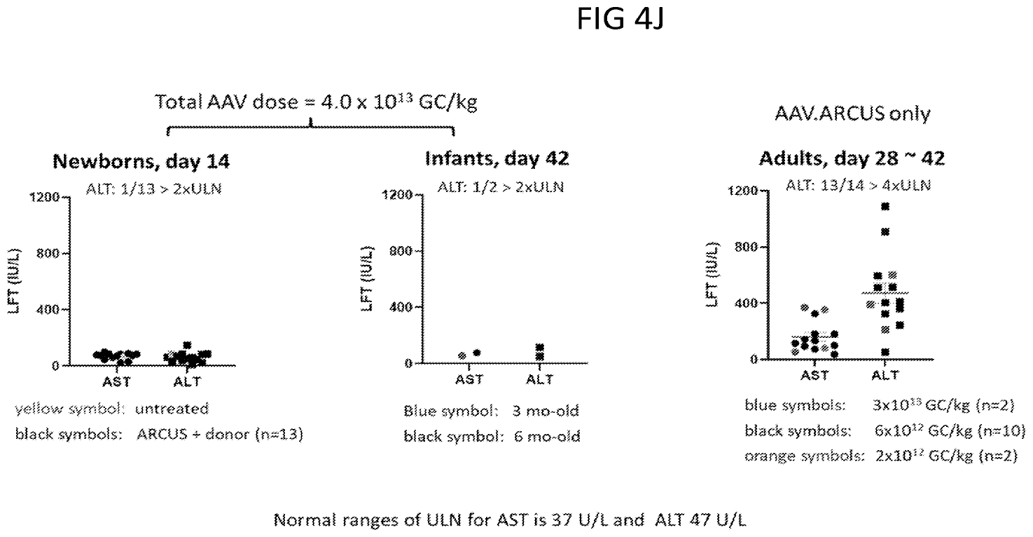

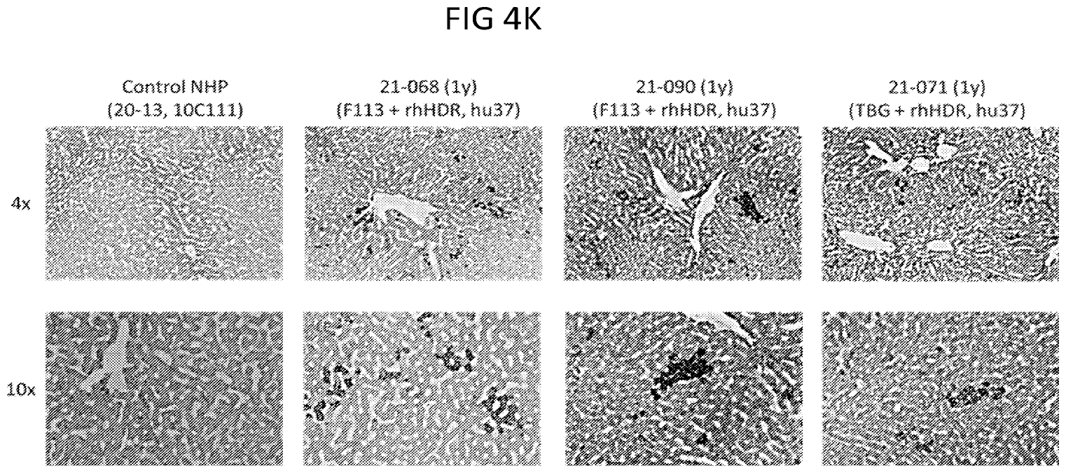

FIG. 1A shows a timeline for the study provided herein. FIG. 1B provides information on the dosing groups, while the vector schematic is shown in FIG. 2. Newborn NHPs (1-16 days) were administered ARCUS2 nuclease vectors, and donor vectors having HDR arms of varying length—500 bp arm or short HDR arm. FIG. 3 is a summary table showing data from the experiment. All 14 newborn macaques tolerated vector infusions well (i.e., no apparent clinical sequelae) and gained weight over time (FIG. 4E). Liver enzyme levels were within the normal range except for transient and modest elevation of ALT levels in some animals on Day 14 (FIG. 4B). Liver inflammation in newborn and infant NHPs was substantially reduced as compared to adult NHPs administered AAV.Arcus only (FIG. 4J). Pancreas was the only non-liver tissue in which any indels were identified (FIG. 4I).

Analysis on the Day 0 plasma samples collected from the newborns prior to dosing showed 3 animals (21-111, 21-113, 21-122) had high levels (>400) of binding antibodies to AAVrh79 (FIG. 3). These pre-existing anti-AAVrh79 antibodies would block AAV gene transfer.

PCSK9 levels were followed in all newborn animals including the donor-only control animals over time. PCSK9 levels on Day 0 varied between the newborns (FIG. 4A). Nine animals showed a trend of reduced PCSK9 levels post vector administration including one donor-only control animal, while the while the remaining five animals showed persistent or transient elevation of PCSK9 levels post dosing FIG. 4A).

On Day 84 and at 1 year, liver biopsies were performed. Transduction efficiencies of hOTC in liver were evaluated by dual ISH with hOTC- and ARCUS-specific probes to detect transgene mRNA, and by OTC immunofluorescence to detect human OTC protein, followed by quantification on scanned slides (FIG. 4C—ISH; FIG. 4D-4F). The three animals (21-111, 21-113, and 21-122) with pre-existing anti-AAVrh79 binding antibodies at the time of dosing did not show any OTC-positive hepatocytes by both methods. The two donor-only control animals continued to show low level (<2%) of hOTC transduction. The highest transduction efficiencies (27.8 and 23.6% by ISH) were detected in the two animals that received a co-administration of AAVrh79.TBG.PI.ARCUS.WPRE.bGH and AAVrh79.rhHDR.TBG.hOTCco.bGH donor vectors (GTP-506). Positive hOTC-expressing hepatocytes were also found to be present in clusters. These levels are above the threshold for substantially benefitting patients, which is ˜5% OTC-expressing cells. The data demonstrate that driving ARCUS from the strong TBG promoter and long homology arms in the donor vector yielded greatest editing. In addition, similarly high levels of editing observed with same ARCUS vector and same homology arms in the F9 donor vector in NB studies. Mouse OTCD pharmacology studies demonstrated consistently high editing with donors containing different homology arms including the mouse correlate of the long homology arm vector in this study. The level of editing NB NHPs with GTP-506 is enough to show strong therapeutic effect.

Molecular analysis on the Day 84 liver biopsy samples from each animal were performed to measure transgene copy numbers per diploid genome, mRNA expression levels, on-target editing, and off-target editing (FIG. 4H). Consistent with the transduction efficiency analyses, the two animals (21-157 and 21-175) in Group 6 had the highest hOTC vector GC (FIG. 4F), hOTC mRNA (FIG. 4G), and on-target indel % (FIG. 4H). The ARCUS vector GC in animals were 2-fold to 7-fold lower than the hOTC vector GC, while ARCUS mRNA levels were 23-fold and 765-fold lower than the hOTC mRNA levels (FIGS. 4F and 4G).

Off-target (OT) activity evaluated by ITR-seq identified 2 to 40 potential off-targets in the Day 84 liver biopsy samples in this study. Some off-target sites were detected in multiple animals, including animals dosed with a human factor IX (hfX) donor vector. Off-target editing was further characterized by amplicon-seq on the potential off-target sites. FIG. 14A provides a list of the off-target sites, along with the chromosomal location and best match to off-target consensus sequence. FIG. 14B is a graph showing indel percentages for OT1-OT10. Editing on OT1, OT4, and OT5 were significantly higher in ARCUS+donor animals than in non-nuclease controls. Off target editing was lower in newborn/infants than in adult NHPs.

The growing livers of newborn/infant primates are highly receptive to site-directed insertion of donor genes. The levels of OTC consistently exceed the 5% therapeutic threshold, which effect appears highly durable. Further, newborn/infant primates are tolerant to toxicity of systemic AAV. There was an excellent correlation of dosing and site-specific integration when comparing NHPs and a mouse model of OTCD.

In summary, we identified an ARCUS vector and hOTCco donor vector combination that when co-administered into newborn macaques could achieve 12-18.6% transduction efficiency in liver at 3 months post dosing, both higher than the threshold for substantially benefitting patients, which is ˜5% OTC-expressing hepatocytes. Animals in this study are being followed for long-term efficiency and safety evaluation. A second liver biopsy was performed at 1 year post dosing to evaluate the stability of hOTC transduction, histopathology in liver, and on- and off-targeting in liver.

### Example 3—PCSK9-hE7-KI Mouse Model

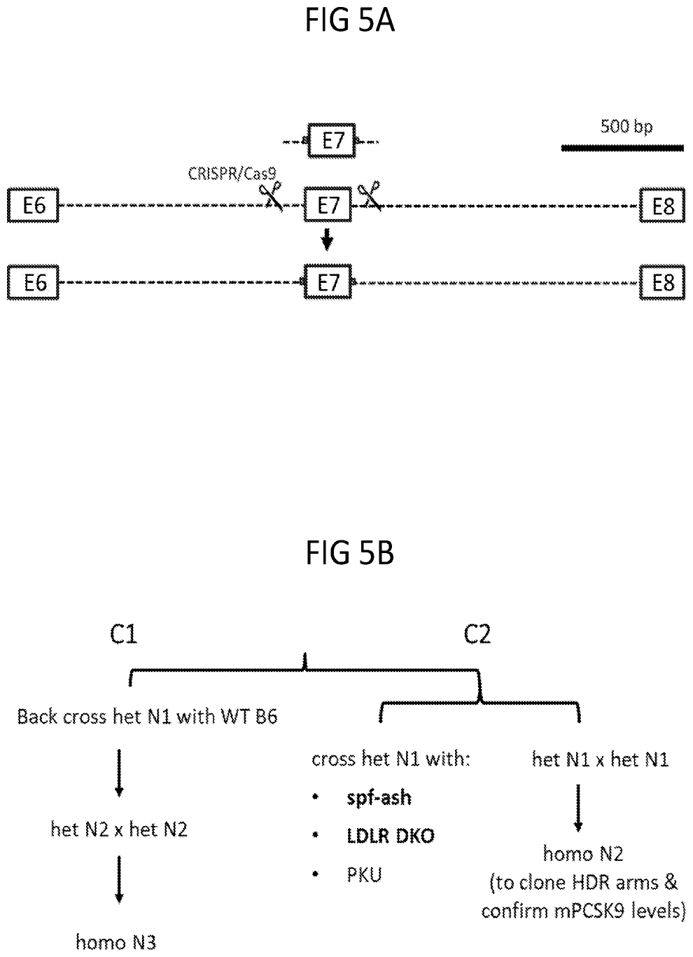

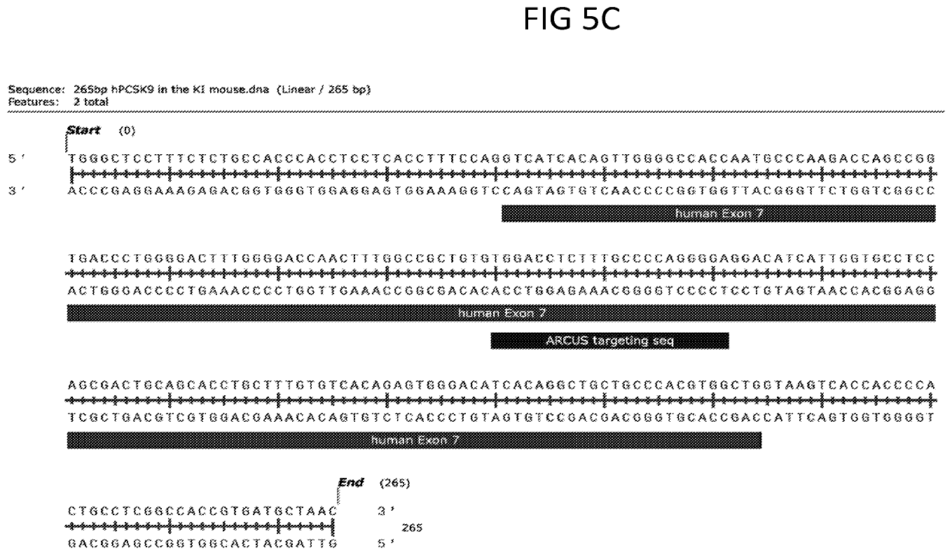

Since the ARCUS targeting sequence in human and macaque PCSK9 gene is not conserved with the murine Pcsk9 gene, we cannot use ARCUS for genome editing in the genomic locus in mouse. Therefore, we commissioned The Jackson Laboratory to generate a knock-in mouse model that replaces a region including the exon 7 of the murine Pcsk9 gene with a region of human PCSK9 gene containing exon 7, named PCSK9-hE7-KI mouse (FIG. 5A-5C). This model can be used for evaluation of in vivo genome editing and gene targeting efficiency. We then crossed PCSK9-hE7-KI mouse with sparse fur ash (spf^{ash}) mouse. spf^{ash} mice have a G to A point mutation at the splice donor site at the end of exon 4 of the Otc gene, which leads to abnormal splicing of Otc mRNA and a 20-fold reduction in both OTC mRNA and protein expression (Hodges and Rosenberg. 1989). Affected animals have 5-10% residual OTC activity and can survive on a chow diet, but they develop hyperammonia that can be lethal when on a high-protein diet (Yang et al., 2016).

The PCSK9-hE7-KI.spf^{ash} mouse model can be used for evaluation of the efficacy of in vivo gene targeting of human OTC and demonstration of correlations of targeting efficiency and efficacy. However, due to the small size of neonatal mice, evaluation of blood clinical pathology and clinical efficacy of gene targeting can only be performed after mice are weaned, once they have reached sufficient body weight, and as a terminal procedure.

FIG. 6 shows sequence alignment of 265 bp sequence represents the human PCSK9 sequence of the PCSK9-hE7 knock-in allele, mouse PCSK9 (mPCSK9) and rhesus macaques PCSK9 (rhPCSK9). There are 6 mismatches between human and rhesus sequences in this 265 bp region. Rodent and primate sequences diverge beyond this window due to insertion of various LINE and LTR. A 2 amino acid difference in exon 7 between human and mouse is present. The hE7-KI mouse is expressing normal levels of mPCSK9 by as measured ELISA.

### Example 4—In vivo OTC Gene Targeting to the Pcsk9 Locus in Pcsk9-hE7-KI.spf^{ash} Pups

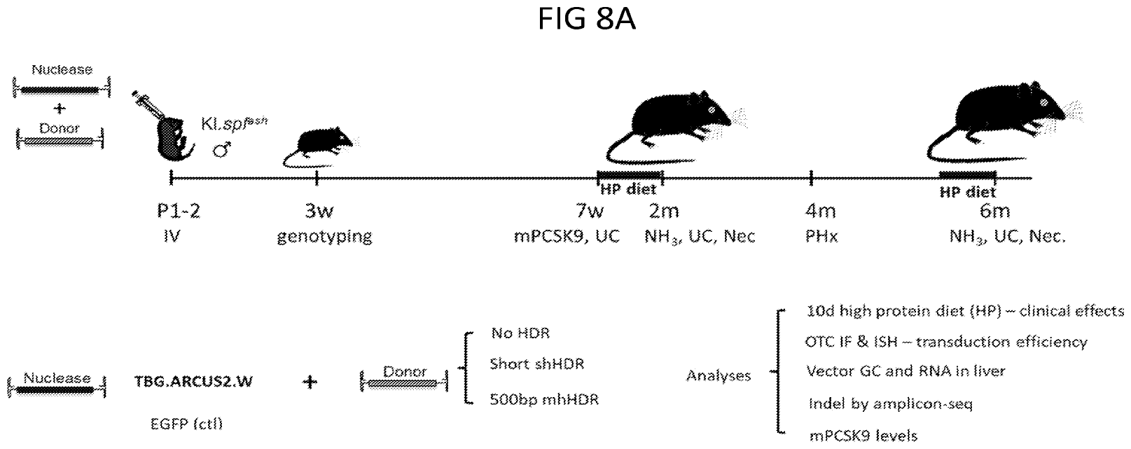

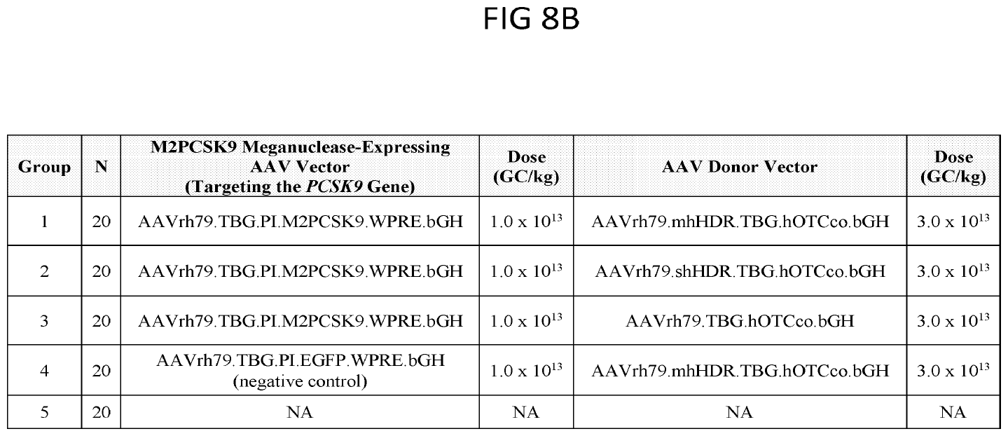

This non-GLP-compliant pharmacology study assesses whether ARCUS meganuclease-mediated knock-in of the human OTC gene in newborn PCSK9-hE7-KI.spf^{ash} mice can achieve therapeutic human OTC expression in the target tissue for treatment of OTC deficiency (liver) following a single co-administration of an ARCUS nuclease-expressing vector and a human OTC donor vector via the intended clinical ROA (IV). A schematic of the experimental design is shown in FIG. 8A.

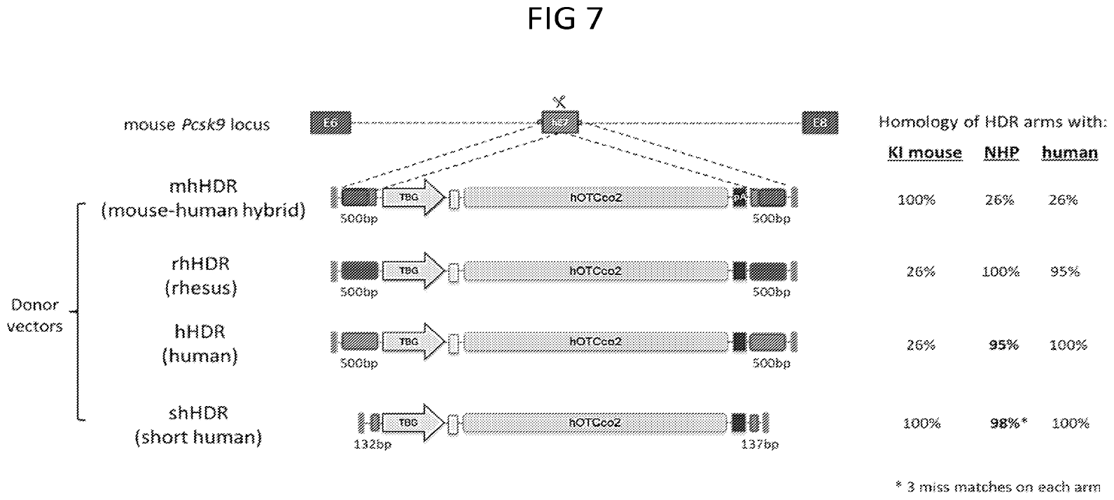

On Day 0, newborn (PND 1-2) male PCSK9-hE7-KI.spf^{ash} mice were IV co-administered an AAVrh79 vector expressing ARCUS meganuclease (AAVrh79.TBG.PI.ARCUS.WPRE.bGH) at a dose of 1.0×10¹³ GC/kg in combination with one of three different AAVrh79 hOTCco donor vectors at a dose of 3.0×10¹³ GC/kg (FIG. 8B). The ARCUS meganuclease-expressing vector evaluated in this study (AAVrh79.TBG.PI.ARCUS.WPRE.bGH) was identical to the lead clinical candidate, while each hOTCco donor vector was identical to the lead clinical candidate except for the HDR arms. Specifically, while the clinical candidate includes a long version of the human HDR sequence (AAVrh79.hHDR.TBG.hOTCco.bGH), the hOTCco donor vectors assessed in this study included a mouse-human hybrid HDR sequence (AAVrh79.mhHDR.TBG.hOTCco.bGH), a shorter version of the human HDR sequence (AAVrh79.shHDR.TBG.hOTCco.bGH), or no HDR sequence (AAVrh79.TBG.hOTCco.bGH). FIG. 7 shows a comparison of the homology of the HDR arms with human, knock in mouse and NHP sequences. As a negative control, additional age-matched PCSK9-hE7-KI.spf^{ash} mice were administered an AAVrh79 vector expressing no meganuclease (AAVrh79.TBG.PI.EGFP.WPRE.bGH) in combination with AAVrh79.mhHDR.TBG.hOTCco.bGH.

In-life evaluations include viability monitoring performed daily, body weight measurements, assessment of plasma PCSK9, and plasma NH₃ and urine orotic acid levels following the high protein diet challenge, and a partial hepatectomy at Day 120 to evaluate the stability of human OTC transduction following two-third partial hepatectomy. On Days 49 and 170, a subgroup from each cohort is challenged with a 10-day high protein diet followed by necropsy at the end of the challenge. At necropsy, livers are collected to evaluate the knock-in of the human OTC gene, including assessment of human OTC mRNA expression (in situ hybridization), OTC protein expression (immunostaining), and OTC enzyme activity assessed by staining and/or an enzyme activity assay. Liver DNA is isolated to assess on-target editing (amplicon-seq, Oxford nanopore long-read sequencing) and evaluate vector genome copies.

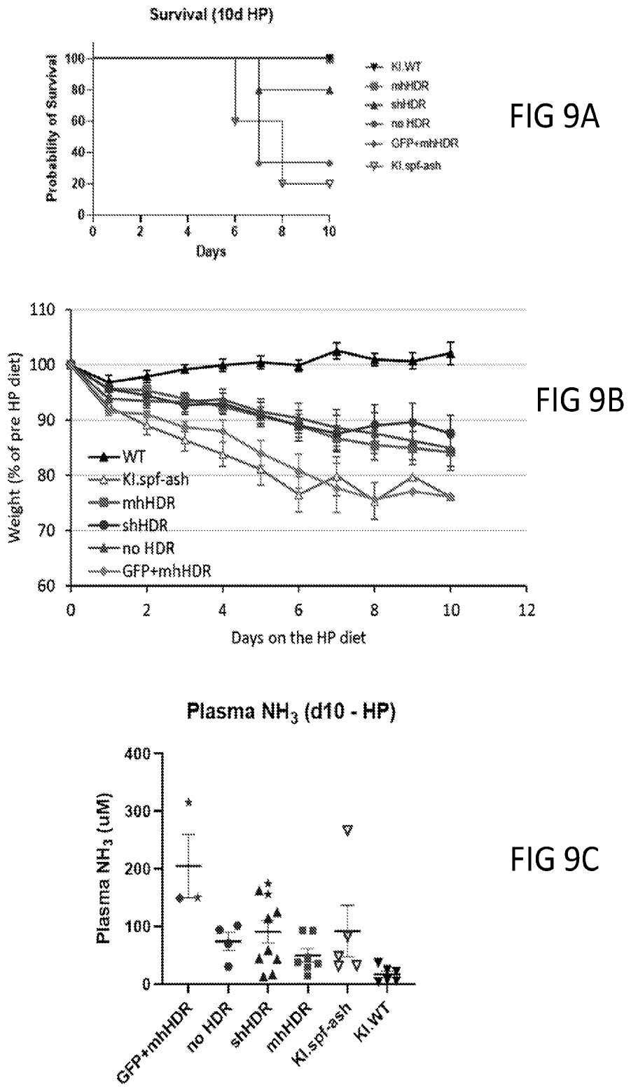

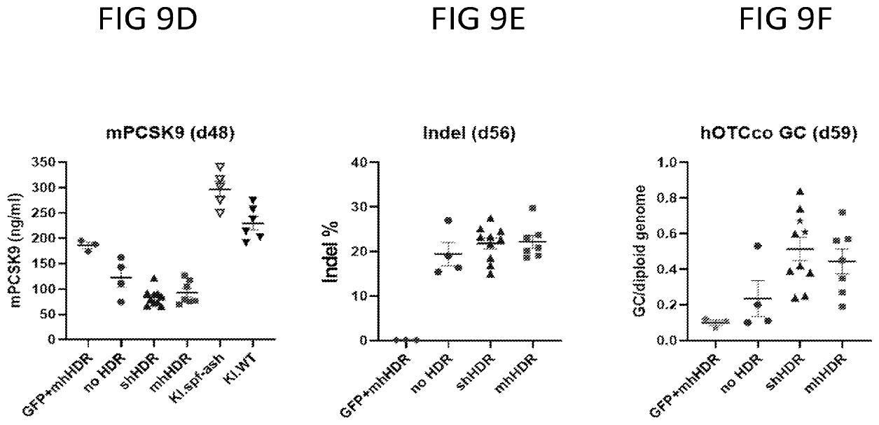

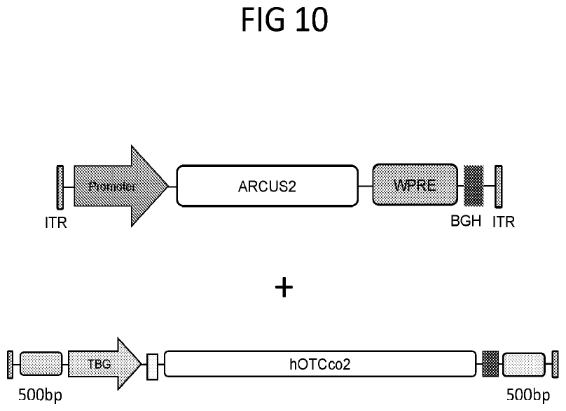

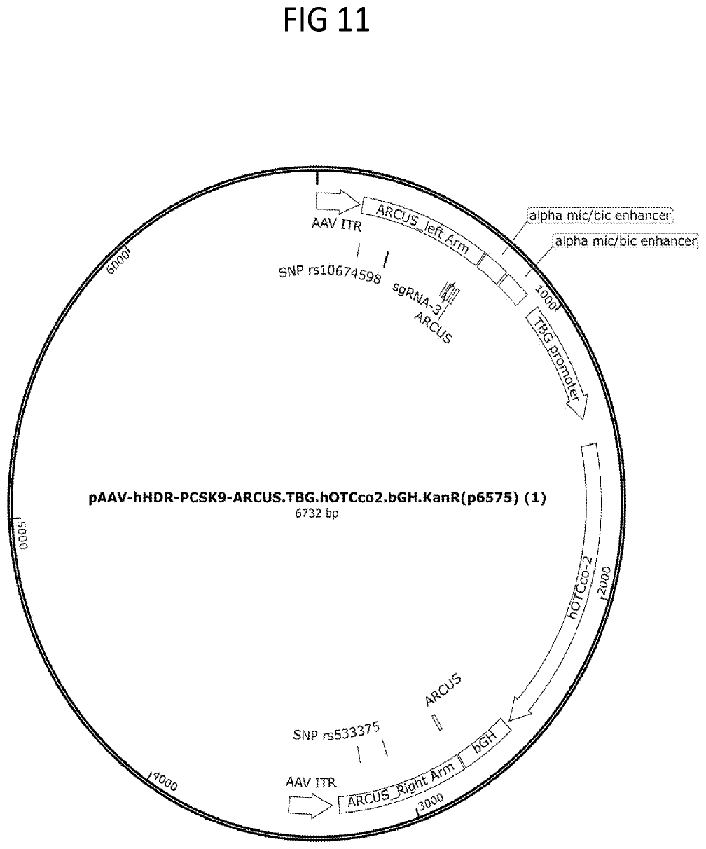

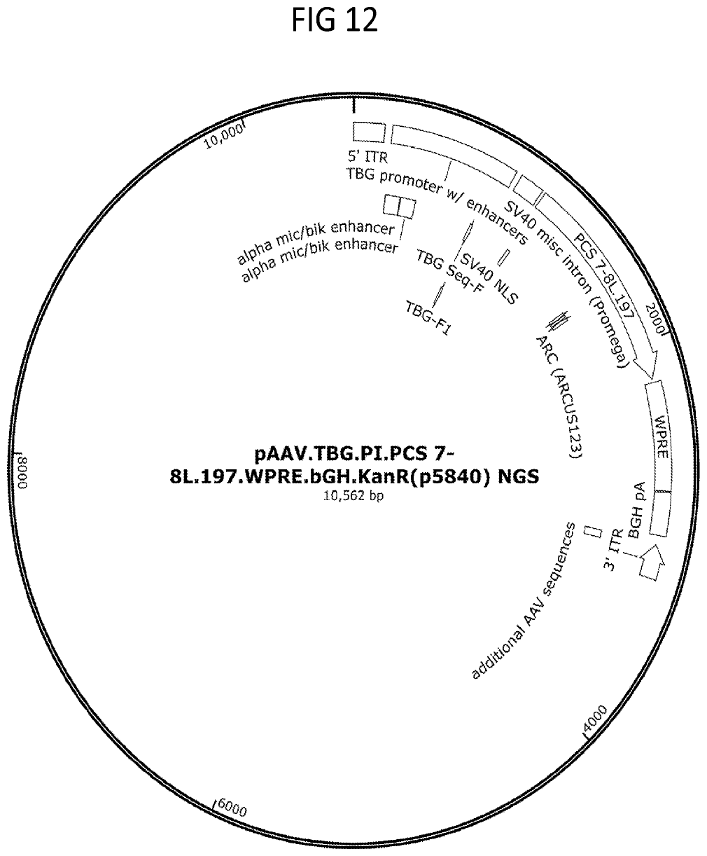

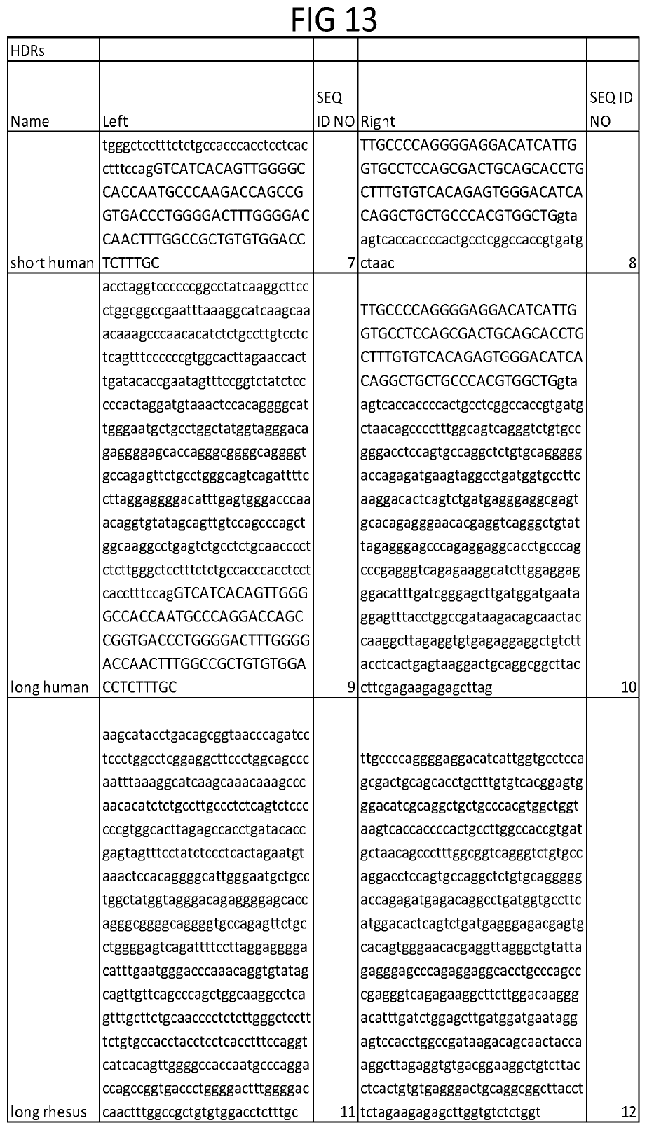

Mice dosed with vectors having the mhHDR arms show survival equivalent to wild type mice, with shHDR-treated mice achieving 80% viability after the 10-day high protein diet challenge (FIG. 9A). All treated mice maintained weight better than KI-spf-ash untreated mice (FIG. 9B). Plasma ammonia levels of mHDR-treated mice were markedly reduced as compared to untreated mice (FIG. 9C).

mPCSK9 levels were measured at date 48 and all treated mice showed a reduction (FIG. 9D). Indel percentage was fairly consistent across HDR types (FIG. 9E). hOTC levels were increased in mice treated with shHDR and mhHDR (FIG. 9F).

### Example 5—Assessing the Efficacy and Determining the Minimum Effective Dose in Pcsk9-hE7-KI.Spf^{ash} Pups

This planned GLP-compliant pharmacology study aims to evaluate the efficacy and determine the MED of IV-administered AAV in the newborn PCSK9-hE7-KI.spf^{ash} mouse model. The AAVrh79 vector expressing ARCUS meganuclease (AAVrh79.TBG.PI.ARCUS.WPRE.bGH) will be the toxicological vector lot that will be manufactured for the planned GLP-compliant toxicology study. Instead of utilizing the test article, which includes a long version of the human HDR sequence (AAVrh79.hHDR.TBG.hOTCco.bGH), this study will utilize the hOTCco donor vector that includes the mouse-human hybrid HDR sequence (AAVrh79.mhHDR.TBG.hOTCco.bGH). This vector will be manufactured in a comparable method to that of the toxicological vector lot of the clinical candidate. We have chosen to use the mouse-human hybrid HDR sequence within the donor vector (AAVrh79.mhHDR.TBG.hOTCco.bGH) for this study to enable us to effectively study the pharmacology of this approach where the donor sequence is directly homologous to the sequence in the PCSK9-hE7-KI.spf^{ash} mice.

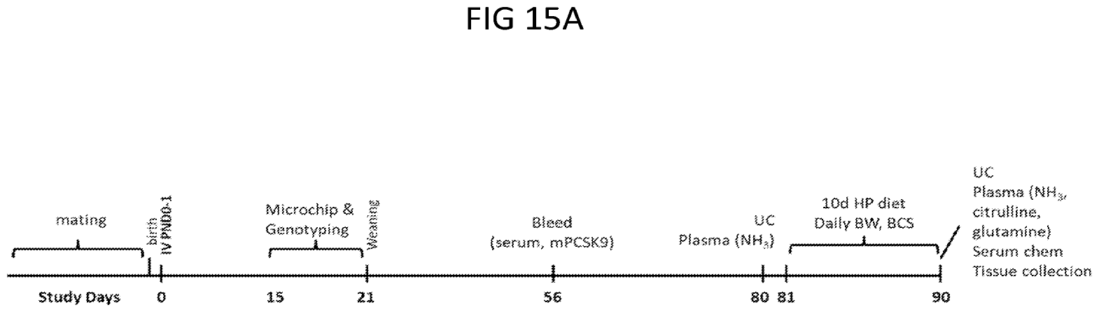

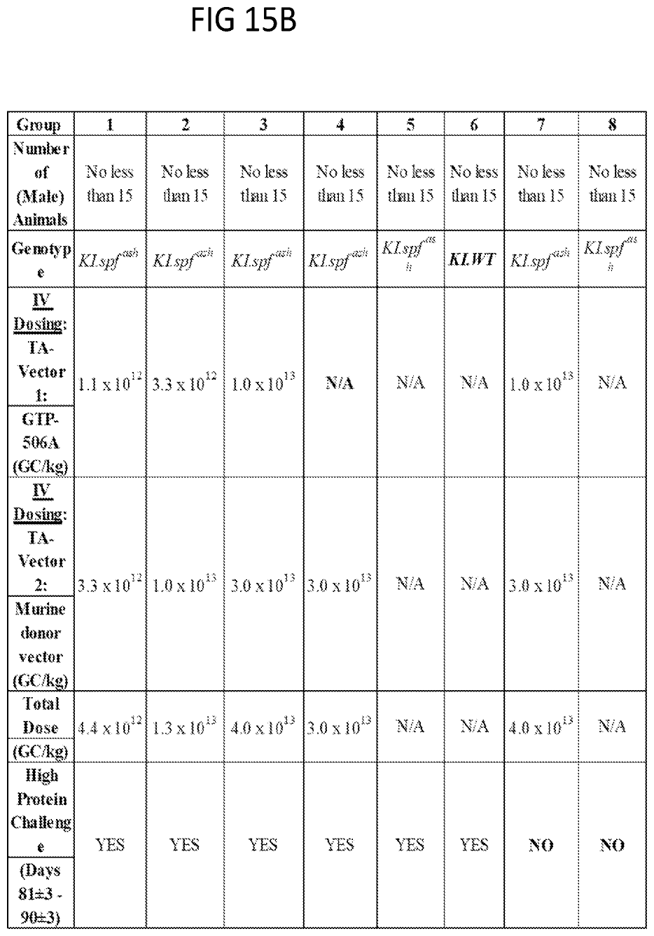

This study will evaluate N=60 neonatal (PND 1-2) newborn PCSK9-hE7-KI.spf^{ash} mice and N=15 age-matched male PCSK9-hE7-KI.WT (wild type) as controls. The study will include one necropsy time point (90 days) (See study design in FIG. 15A). For efficacy evaluation, mice will be challenged by a 10-day course of a high protein diet from Day 81 to Day 90. Survival, body conditions, and biomarker changes will be evaluated. The mice will receive one of three dosage levels of the test article (1.0×10¹² GC/kg nuclease vector and 3.0×10¹² GC/kg donor vector, 3.3×10¹² GC/kg nuclease vector and 1.0×10¹³ GC/kg donor vector, or 1.0×10¹³ GC/kg nuclease vector and 3.0×10¹³ GC/kg; N=20 per dose) or vehicle (phosphate-buffered saline [PBS]; N=20). The dosages are noted in FIG. 15B.

In-life assessments include daily viability checks, monitoring for survival, body weight measurements, assessment of serum PCSK9 levels, plasma NH₃ and urine orotic acid levels following high protein diet challenge. Necropsies will be performed on Day 90. At necropsy, blood will be collected for CBC/differentials and serum clinical chemistry analysis. A list of tissues will be collected for histopathological evaluation. Liver will be collected to evaluate the knock-in of the human OTC gene, including assessment of human OTC mRNA expression (in situ hybridization), OTC protein expression (immunostaining), and OTC enzyme activity assessed by staining and/or an enzyme activity assay. Liver DNA will also be isolated to assess on-target editing (amplicon-seq) and evaluate vector genome copies.

The MED will be determined based upon analysis of survival following the high protein diet, plasma NH₃ levels at the end of the high protein diet challenge human OTC mRNA and protein expression, OTC enzyme activity, and on-target editing of AAV-treated newborn PCSK9-hE7-KI.spf^{ash} compared to vehicle-treated newborn PCSK9-hE7-KI.spf^{ash} control mice.

### Example 8—Toxicology Study in Pcsk9-hE7-KI.Spf^{ash} Pups

A 6-month GLP-compliant safety study will be conducted in newborn (PND 1-2) PCSK9-hE7-KI.spf^{ash} mice to investigate the safety, tolerability, pharmacology, and pharmacokinetics of the test article following IV co-administration. Interim analyses, including on-target editing, off-target editing, transgene expression, and histopathological analyses, will be performed on Day 60 and Day 180 as these time points will allow sufficient time for the nuclease-dependent gene insertion to have reached stable plateau levels following administration. Newborn PCSK9-hE7-KI.spf^{ash} mice will receive one of three dose levels of the test article (1.0×10¹² GC/kg nuclease vector and 3.0×10¹² GC/kg donor vector, 3.3×10¹² GC/kg nuclease vector and 1.0×10¹³ GC/kg donor vector, or 1.0×10¹³ GC/kg nuclease vector and 3.0×10¹³ GC/kg; N=20 per dose) or vehicle (phosphate-buffered saline [PBS]; N=20). After the test article or vehicle administration, in-life evaluations will include clinical observations to monitor daily for signs of distress and abnormal behavior, body weight measurements, and blood clinical serum chemistry (specifically ALT, AST, and total bilirubin).

On Day 60 after test article administration, cohorts 1, 3, 5, and 7 of will be euthanized, and a histopathological analysis will be performed on a comprehensive list of tissues, including, but not limited to, brain, spinal cord, heart, liver, spleen, kidney, lungs, reproductive organs, adrenal glands, and lymph nodes. Organs will be weighed as appropriate.

Liver samples will be collected and analyzed for on-target editing by amplicon-seq and AMP-seq, off-target editing by ITR-seq and amplicon-seq, vector biodistribution, and transgene expression. In liver samples, biodistribution will be evaluated by PCR and meganuclease RNA expression will be analyzed by RT-PCR will be performed. Highly perfused organs will be analyzed for meganuclease RNA and tissues with detectable expression of meganuclease RNA will be evaluated for on-target editing by amplicon-seq. Tissues with detectable on-target editing will be further evaluated for off-target editing.

For vector biodistribution, qPCR detection specific to the transgenes of the dual vectors, ARCUS and hOTCco, will be developed. The efficiency, linearity, precision, reproducibility and limit of detection of the assays will be assessed using the AAV cis plasmids as surrogates of target sequence. The lower limit of quantification (LLOQ) of the assays will be determined prior to the assay on test tissues or excreta initiated. A qualification plan will be implemented to bridge the transgene specific assays to the qualification studies conducted previously. The matrix tested will include intended target, liver for biodistribution. The matrix effect will be further evaluated based on the recovery of spiked target controls from all samples tested in the course of biodistribution studies as well as the data subtracted from the qualification studies conducted previously.

### Example 9—Toxicology Study in Infant (6-9 Week Old) NHP

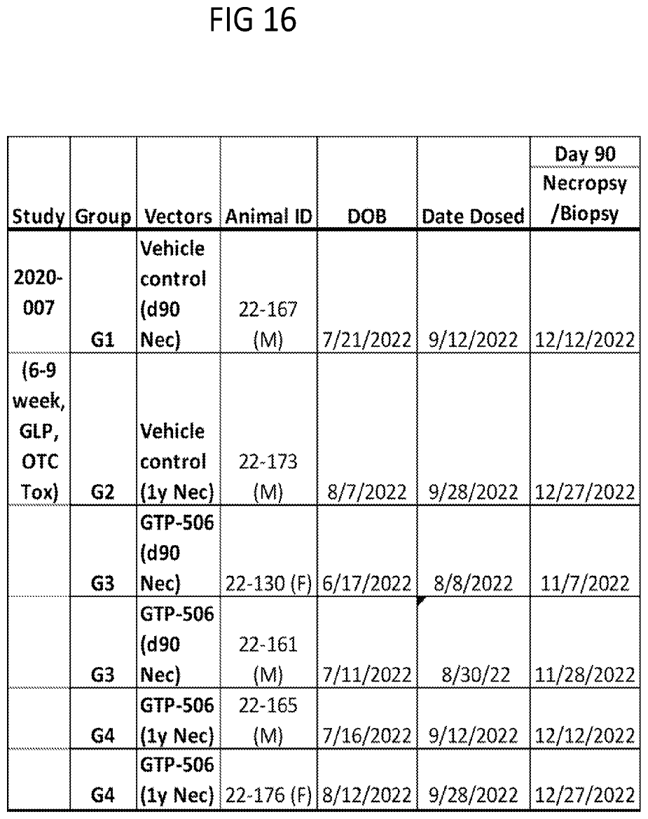

A 1 year GLP-compliant safety study will be conducted in infant (6-9 week) rhesus macaques to investigate the safety, tolerability, pharmacology, and pharmacokinetics of the test article following IV co-administration. Interim analyses, including on-target editing, off-target editing, transgene expression, and histopathological analyses, will be performed on Day 90 as this time point will allow sufficient time for the nuclease-dependent gene insertion to have reached stable plateau levels following administration. Infant rhesus macaques will receive 1.0×10¹³ GC/kg nuclease vector and 3.0×10¹³ GC/kg; N=4) or vehicle (phosphate-buffered saline [PBS]/surfactant; N=2). After the test article or vehicle administration, in-life evaluations will include clinical observations to monitor daily for signs of distress and abnormal behavior, body weight measurements, and blood clinical serum chemistry (specifically ALT, AST, and total bilirubin). The study design is shown in FIG. 16.

On Day 90 after test article administration, one animal in the vehicle control group and Group 3 of will be euthanized, and a histopathological analysis will be performed on a comprehensive list of tissues, including, but not limited to, brain, spinal cord, heart, liver, spleen, kidney, lungs, reproductive organs, adrenal glands, and lymph nodes. Organs will be weighed as appropriate. At 90 days, the remaining animals will receive a liver biopsy.

Liver samples will be collected and analyzed for on-target editing by amplicon-seq and AMP-seq, off-target editing by ITR-seq and amplicon-seq, vector biodistribution, and transgene expression. In liver samples, biodistribution will be evaluated by PCR and meganuclease RNA expression will be analyzed by RT-PCR will be performed. Highly perfused organs will be analyzed for meganuclease RNA and tissues with detectable expression of meganuclease RNA will be evaluated for on-target editing by amplicon-seq. Tissues with detectable on-target editing will be further evaluated for off-target editing.

For vector biodistribution, qPCR detection specific to the transgenes of the dual vectors, ARCUS and hOTCco, will be developed. The efficiency, linearity, precision, reproducibility and limit of detection of the assays will be assessed using the AAV cis plasmids as surrogates of target sequence. The lower limit of quantification (LLOQ) of the assays will be determined prior to the assay on test tissues or excreta initiated. A qualification plan will be implemented to bridge the transgene specific assays to the qualification studies conducted previously. The matrix tested will include intended target, liver for biodistribution. The matrix effect will be further evaluated based on the recovery of spiked target controls from all samples tested in the course of biodistribution studies as well as the data subtracted from the qualification studies conducted previously.

All documents cited in this specification are incorporated herein by reference, as are sequences and text of the Sequence Listing filed herewith are incorporated by reference. US Provisional Patent Application Nos. 63/301,917 filed Jan. 21, 2022, 63/331,384, filed Apr. 15, 2022, 63/364,861, filed May 17, 2022, 63/370,049, filed Aug. 1, 2022, and 63/379,067, filed Oct. 11, 2022 are each incorporated by reference in their entirety. While the invention has been described with reference to particular embodiments, it will be appreciated that modifications can be made without departing from the spirit of the invention. Such modifications are intended to fall within the scope of the appended claims.

### REFERENCES

1. Al-Zaidy S., Pickard A. S., Kotha K., Alfano L. N., Lowes L., Paul G., Church K., Lehman K., Sproule D. M., Dabbous O., Maru B., Berry K., Arnold W. D., Kissel J. T., Mendell J. R., & Shell R. (2019). “Health Outcomes in Spinal Muscular Atrophy Type 1 Following Avxs-101 Gene Replacement Therapy.” Pediatr Pulmonol. 54(2):179-185.
2. Anderson A., Gropman A., Le Mons C., Stratakis C., & Gandjbakhche A. (2020). “Evaluation of Neurocognitive Function of Prefrontal Cortex in Ornithine Transcarbamylase Deficiency.” Mol Genet Metab. 129(3):207-212.
3. Arnould S., Delenda C., Grizot S., Desseaux C., Paques F., Silva G. H., & Smith J. (2011). “The I-Crei Meganuclease and Its Engineered Derivatives: Applications from Cell Modification to Gene Therapy.” Protein Eng Des Sel. 24(1-2):27-31.
4. Audentes. (2018). “Audentes Announces Positive Interim Data from First Dose Cohort of Aspiro, a Phase 1/2 Clinical Trial of At132 in Patients with X-Linked Myotubular Myopathy.” Retrieved May 15, 2021, from https://www.prnewswire.com/news-releases/audentes-announces-positive-interim-data-from-first-dose-cohort-of-aspiro-a-phase-12-clinical-trial-of-at132-in-patients-with-x-linked-myotubular-myopathy-300577455.html
5. AveXis. (2019). “Zolgensma® (Onasemnogene Abeparvovec-Xioi) Suspension, for Intravenous Infusion.” Retrieved May 15, 2021, from https://www.fda.Qov/media/126109/download.
6. Bao X. R., Pan Y., Lee C. M., Davis T. H., & Bao G. (2021). “Tools for Experimental and Computational Analyses of Off-Target Editing by Programmable Nucleases.” Nat Protoc. 16(1):10-26.
7. Batshaw M. L., Tuchman M., Summar M., & Seminara J. (2014). “A Longitudinal Study of Urea Cycle Disorders.” Mol Genet Metab. 113(1-2):127-30.
8. Bell P., Moscioni A. D., McCarter R. J., Wu D., Gao G., Hoang A., Sanmiguel J. C., Sun X., Wivel N. A., Raper S. E., Furth E. E., Batshaw M. L., & Wilson J. M. (2006). “Analysis of Tumors Arising in Male B6c3f1 Mice with and without Aav Vector Delivery to Liver.” Mol Ther. 14(1):34-44.
9. Bell P., Wang L., Lebherz C., Flieder D. B., Bove M. S., Wu D., Gao G. P., Wilson J. M., & Wivel N. A. (2005). “No Evidence for Tumorigenesis of Aav Vectors in a Large-Scale Study in Mice.” Mol Ther. 12(2):299-306.
10. Bennett E. P., Petersen B. L., Johansen I. E., Niu Y., Yang Z., Chamberlain C. A., Met O., Wandall H. H., & Frodin M. (2020). “Indel Detection, the ‘Achilles Heel’ of Precise Genome Editing: A Survey of Methods for Accurate Profiling of Gene Editing Induced Indels.” Nucleic Acids Res. 48(21):11958-11981.
11. Berraondo P., Martini P. G. V., Avila M. A., & Fontanellas A. (2019). “Messenger Rna Therapy for Rare Genetic Metabolic Diseases.” Gut. 68(7):1323-1330.
12. Berry G. T. & Steiner R. D. (2001). “Long-Term Management of Patients with Urea Cycle Disorders.” J Pediatr. 138(1 Suppl):556-60; discussion S60-1.
13. Bevan A. K., Duque S., Foust K. D., Morales P. R., Braun L., Schmelzer L., Chan C. M., McCrate M., Chicoine L. G., Coley B. D., Porensky P. N., Kolb S. J., Mendell J. R., Burghes A. H., & Kaspar B. K. (2011). “Systemic Gene Delivery in Large Species for Targeting Spinal Cord, Brain, and Peripheral Tissues for Pediatric Disorders.” Mol Ther. 19(11):1971-80.
14. Breton C., Clark P. M., Wang L., Greig J. A., & Wilson J. M. (2020). “Itr-Seq, a Next-Generation Sequencing Assay, Identifies Genome-Wide DNA Editing Sites in Vivo Following Adeno-Associated Viral Vector-Mediated Genome Editing.” BMC genomics. 21(1):1-12.
15. Breton C., Furmanak T., Avitto A. N., Smith M. K., Latshaw C., Yan H., Greig J. A., & Wilson J. M. (2021). “Increasing the Specificity of Aav-Based Gene Editing through Self-Targeting and Short-Promoter Strategies.” Mol Ther. 29(3):1047-1056.
16. Calcedo R., Vandenberghe L. H., Gao G., Lin J., & Wilson J. M. (2009). “Worldwide Epidemiology of Neutralizing Antibodies to Adeno-Associated Viruses.” J Infect Dis. 199(3):381-90.
17. Caldovic L., Abdikarim I., Narain S., Tuchman M., & Morizono H. (2015). “Genotype-Phenotype Correlations in Ornithine Transcarbamylase Deficiency: A Mutation Update.” J Genet Genomics. 42(5):181-94.
18. Castle M. J., Perlson E., Holzbaur E. L., & Wolfe J. H. (2014). “Long-Distance Axonal Transport of Aav9 Is Driven by Dynein and Kinesin-2 and Is Trafficked in a Highly Motile Rab7-Positive Compartment.” Mol Ther. 22(3):554-566.
19. Chand D. H., Zaidman C., Arya K., Millner R., Farrar M. A., Mackie F. E., Goedeker N. L., Dharnidharka V. R., Dandamudi R., & Reyna S. P. (2021). “Thrombotic Microangiopathy Following Onasemnogene Abeparvovec for Spinal Muscular Atrophy: A Case Series.” The Journal of Pediatrics. 231:265-268.
20. Chandler R. J., LaFave M. C., Varshney G. K., Trivedi N. S., Carrillo-Carrasco N., Senac J. S., Wu W., Hoffmann V., Elkahloun A. G., Burgess S. M., & Venditti C. P. (2015). “Vector Design Influences Hepatic Genotoxicity after Adeno-Associated Virus Gene Therapy.” J Clin Invest. 125(2):870-80.
21. Colella P., Ronzitti G., & Mingozzi F. (2018). “Emerging Issues in Aav-Mediated in Vivo Gene Therapy.” Mol Ther Methods Clin Dev. 8:87-104.
22. Couto L., Parker A., & Gordon J. W. (2004). “Direct Exposure of Mouse Spermatozoa to Very High Concentrations of a Serotype-2 Adeno-Associated Virus Gene Therapy Vector Fails to Lead to Germ Cell Transduction.” Hum Gene Ther. 15(3):287-91.
23. Cunningham S. C., Dane A. P., Spinoulas A., Logan G. J., & Alexander I. E. (2008). “Gene Delivery to the Juvenile Mouse Liver Using Aav2/8 Vectors.” Mol Ther. 16(6):1081-8.
24. Cunningham S. C., Spinoulas A., Carpenter K. H., Wilcken B., Kuchel P. W., & Alexander I. E. (2009). “Aav2/8-Mediated Correction of Otc Deficiency Is Robust in Adult but Not Neonatal Spf(Ash) Mice.” Mol Ther. 17(8):1340-6.
25. Dabbous O., Maru B., Jansen J. P., Lorenzi M., Cloutier M., Guerin A., Pivneva I., Wu E. Q., Arjunji R., Feltner D., & Sproule D. M. (2019). “Survival, Motor Function, and Motor Milestones: Comparison of Avxs-101 Relative to Nusinersen for the Treatment of Infants with Spinal Muscular Atrophy Type 1.” Adv Ther. 36(5):1164-1176.
26. Dalwadi D. A., Torrens L., Abril-Fornaguera J., Pinyol R., Willoughby C., Posey J., Llovet J. M., Lanciault C., Russell D. W., Grompe M., & Naugler W. E. (2021). “Liver Injury Increases the Incidence of Hcc Following Aav Gene Therapy in Mice.” Molecular Therapy. 29(2):680-690.
27. Donsante A., Vogler C., Muzyczka N., Crawford J. M., Barker J., Flotte T., Campbell-Thompson M., Daly T., & Sands M. S. (2001). “Observed Incidence of Tumorigenesis in Long-Term Rodent Studies of Raav Vectors.” Gene Ther. 8(17):1343-6.
28. Favaro P., Downey H. D., Zhou J. S., Wright J. F., Hauck B., Mingozzi F., High K. A., & Arruda V. R. (2009). “Host and Vector-Dependent Effects on the Risk of Germline Transmission of Aav Vectors.” Mol Ther. 17(6):1022-30.
29. Fehse B. & Abramowski-Mock U. (2018). “The Time Is Ripe for Somatic Genome Editing: Nih Program To strengthen Translation.” Mol Ther. 26(3):671-674.
30. Ferla R., Alliegro M., Marteau J. B., Dell'Anno M., Nusco E., Pouillot S., Galimberti S., Valsecchi M. G., Zuliani V., & Auricchio A. (2017). “Non-Clinical Safety and Efficacy of an Aav2/8 Vector Administered Intravenously for Treatment of Mucopolysaccharidosis Type Vi.” Mol Ther Methods Clin Dev. 6:143-158.
31. Fernandez A., Josa S., & Montoliu L. (2017). “A History of Genome Editing in Mammals.” Mamm Genome. 28(7-8):237-246.
32. Flanigan K. M., Simmons T. R., McBride K. L., Kunkler K. L., Alyward S., Couce M. L., Oreiro M. T., Smith N. J. C., Jaensch L., Fuller M., Ruiz J., & Truxal K. V. (2019). “Phase 1/2 Clinical Trial of Systemic Gene Transfer of Scaav9.U1a.Hsgsh for Mps Iiia Demonstrates 2 Years of Safety, Tolerability, and Biopotency.” Molecular Genetics and Metabolism. 126(2):S54.
33. Gao G., Vandenberghe L. H., Alvira M. R., Lu Y., Calcedo R., Zhou X., & Wilson J. M. (2004). “Clades of Adeno-Associated Viruses Are Widely Disseminated in Human Tissues.” J Virol. 78(12):6381-8.
34. George L. A. & Fogarty P. F. (2016). “Gene Therapy for Hemophilia: Past, Present and Future.” Semin Hematol. 53(1):46-54.
35. George L. A., Ragni M. V., Rasko J. E. J., Raffini L. J., Samelson-Jones B. J., Ozelo M., Hazbon M., Runowski A. R., Wellman J. A., Wachtel K., Chen Y., Anguela X. M., Kuranda K., Mingozzi F., & High K. A. (2020). “Long-Term Follow-up of the First in Human Intravascular Delivery of Aav for Gene Transfer: Aav2-Hfixl6 for Severe Hemophilia B.” Molecular Therapy. 28(9):2073-2082.
36. George L. A., Sullivan S. K., Giermasz A., Rasko J. E. J., Samelson-Jones B. J., Ducore J., Cuker A., Sullivan L. M., Majumdar S., Teitel J., McGuinn C. E., Ragni M. V., Luk A. Y., Hui D., Wright J. F., Chen Y., Liu Y., Wachtel K., Winters A., Tiefenbacher S., Arruda V. R., van der Loo J. C. M., Zelenaia O., Takefman D., Carr M. E., Couto L. B., Anguela X. M., & High K. A. (2017). “Hemophilia B Gene Therapy with a High-Specific-Activity Factor Ix Variant.” N Engl J Med. 377(23):2215-2227.
37. Gil-Farina I., Fronza R., Kaeppel C., Lopez-Franco E., Ferreira V., D'Avola D., Benito A., Prieto J., Petry H., Gonzalez-Aseguinolaza G., & Schmidt M. (2016). “Recombinant Aav Integration Is Not Associated with Hepatic Genotoxicity in Nonhuman Primates and Patients.” Mol Ther. 24(6):1100-1105.
38. Godel T., Pham M., Heiland S., Bendszus M., & Baumer P. (2016). “Human Dorsal-Root-Ganglion Perfusion Measured in-Vivo by Mri.” Neuroimage. 141:81-87.
39. Gombash S. E., Cowley C. J., Fitzgerald J. A., Lepak C. A., Neides M. G., Hook K., Todd L. J., Wang G. D., Mueller C., Kaspar B. K., Bielefeld E. C., Fischer A. J., Wood J. D., & Foust K. D. (2017). “Systemic Gene Delivery Transduces the Enteric Nervous System of Guinea Pigs and Cynomolgus Macaques.” Gene Ther. 24(10):640-648.
40. Gordon N. (2003). “Ornithine Transcarbamylase Deficiency: A Urea Cycle Defect.” Eur J Paediatr Neurol. 7(3):115-21.
41. Gray S. J., Nagabhushan Kalburgi S., McCown T. J., & Jude Samulski R. (2013). “Global Cns Gene Delivery and Evasion of Anti-Aav-Neutralizing Antibodies by Intrathecal Aav Administration in Non-Human Primates.” Gene Ther. 20(4):450-9.
42. Greig J. A., Limberis M. P., Bell P., Chen S. J., Calcedo R., Rader D. J., & Wilson J. M. (2017). “Non-Clinical Study Examining Aav8.Tbg.Hldlr Vector-Associated Toxicity in Chow-Fed Wild-Type and Ldlr(+/−) Rhesus Macaques.” Hum Gene Ther Clin Dev. 28(1):39-50.
43. Hanlon K. S., Kleinstiver B. P., Garcia S. P., Zaborowski M. P., Volak A., Spirig S. E., Muller A., Sousa A. A., Tsai S. Q., Bengtsson N. E., Loov C., Ingelsson M., Chamberlain J. S., Corey D. P., Aryee M. J., Joung J. K., Breakefield X. O., Maguire C. A., & Gyorgy B. (2019). “High Levels of Aav Vector Integration into Crispr-Induced DNA Breaks.” Nat Commun. 10(1):44390.
44. Hartung B., Temme O., Neuen-Jacob E., Ritz-Timme S., Hinderhofer K., & Daldrup T. (2016). “Omithine Transcarbamylase Deficiency of a Male Newborn with Fatal Outcome.” Int J Legal Med. 130(3):783-5.
45. Her J. & Bunting S. F. (2018). “How Cells Ensure Correct Repair of DNA Double-Strand Breaks.” J Biol Chem. 293(27):10502-10511.
46. Hinderer C., Bell P., Katz N., Vite C. H., Louboutin J. P., Bote E., Yu H., Zhu Y., Casal M. L., Bagel J., O'Donnell P., Wang P., Haskins M. E., Goode T., & Wilson J. M. (2018a). “Evaluation of Intrathecal Routes of Administration for Adeno-Associated Viral Vectors in Large Animals.” Hum Gene Ther. 29(1):15-24.
47. Hinderer C., Katz N., Buza E. L., Dyer C., Goode T., Bell P., Richman L. K., & Wilson J. M. (2018b). “Severe Toxicity in Nonhuman Primates and Piglets Following High-Dose Intravenous Administration of an Adeno-Associated Virus Vector Expressing Human Smn.” Human gene therapy. 29(3):285-298.
48. Hodges P. E. & Rosenberg L. E. (1989). “The Spfash Mouse: A Missense Mutation in the Ornithine Transcarbamylase Gene Also Causes Aberrant Mrna Splicing.” Proc Natl Acad Sci USA. 86(11):4142-6.
49. Honaramooz A., Megee S., Zeng W., Destrempes M. M., Overton S. A., Luo J., Galantino-Homer H., Modelski M., Chen F., & Blash S. (2008). “Adeno-Associated Virus (Aav)-Mediated Transduction of Male Germ Line Stem Cells Results in Transgene Transmission after Germ Cell Transplantation.” The FASEB Journal. 22(2):374-382.
50. Hordeaux J., Buza E. L., Dyer C., Goode T., Mitchell T. W., Richman L., Denton N., Hinderer C., Katz N., Schmid R., Miller R., Choudhury G. R., Horiuchi M., Nambiar K., Yan H., Li M., & Wilson J. M. (2020). “Aav-Induced Dorsal Root Ganglion Pathology.” Hum Gene Ther.
51. Hordeaux J., Hinderer C., Buza E. L., Louboutin J. P., Jahan T., Bell P., Chichester J. A., Tarantal A. F., & Wilson J. M. (2019). “Safe and Sustained Expression of Human Iduronidase after Intrathecal Administration of Adeno-Associated Virus Serotype 9 in Infant Rhesus Monkeys.” Hum Gene Ther. 30(8):957-966.
52. Hordeaux J., Hinderer C., Goode T., Buza E. L., Bell P., Calcedo R., Richman L. K., & Wilson J. M. (2018a). “Toxicology Study of Intra-Cisterna Magna Adeno-Associated Virus 9 Expressing Iduronate-2-Sulfatase in Rhesus Macaques.” Mol Ther Methods Clin Dev. 10:68-78.
53. Hordeaux J., Hinderer C., Goode T., Katz N., Buza E. L., Bell P., Calcedo R., Richman L. K., & Wilson J. M. (2018b). “Toxicology Study of Intra-Cisterna Magna Adeno-Associated Virus 9 Expressing Human Alpha-L-Iduronidase in Rhesus Macaques.” Mol Ther Methods Clin Dev. 10:79-88.
54. Hordeaux J., Song C., Wielechowski E., Ramezani A., Dyer C., Buza E. L., Chichester J., Bell P., & Wilson J. M. (2021). “Characterization of Acute Toxicity after High-Dose Systemic Adeno-Associated Virus in Nonhuman Primates, Including Impact of Vector Characteristics.” Mol. Ther. 29(4):23.
55. Hordeaux J., Wang Q., Katz N., Buza E. L., Bell P., & Wilson J. M. (2018c). “The Neurotropic Properties of Aav-Php.B Are Limited to C57bl/6j Mice.” Molecular Therapy. 26(3):664-668.
56. Jakob M., Muhle C., Park J., Weiss S., Waddington S., & Schneider H. (2005). “No Evidence for Germ-Line Transmission Following Prenatal and Early Postnatal Aav-Mediated Gene Delivery.” J Gene Med. 7(5):630-7.
57. Jinek M., Chylinski K., Fonfara I., Hauer M., Doudna J. A., & Charpentier E. (2012). “A Programmable Dual-Rna-Guided DNA Endonuclease in Adaptive Bacterial Immunity.” Science. 337(6096):816-21.
58. Kay M. A., Manno C. S., Ragni M. V., Larson P. J., Couto L. B., McClelland A., Glader B., Chew A. J., Tai S. J., Herzog R. W., Arruda V., Johnson F., Scallan C., Skarsgard E., Flake A. W., & High K. A. (2000). “Evidence for Gene Transfer and Expression of Factor Ix in Haemophilia B Patients Treated with an Aav Vector.” Nat Genet. 24(3):257-61.
59. Lakoski S. G., Lagace T. A., Cohen J. C., Horton J. D., & Hobbs H. H. (2009). “Genetic and Metabolic Determinants of Plasma Pcsk9 Levels.” J Clin Endocrinol Metab. 94(7):2537-43.
60. Lee T., Misaki M., Shimomura H., Tanaka Y., Yoshida S., Murayama K., Nakamura K., Fujiki R., Ohara O., Sasai H., Fukao T., & Takeshima Y. (2018). “Late-Onset Omithine Transcarbamylase Deficiency Caused by a Somatic Mosaic Mutation.” Hum Genome Var. 5:22.
61. Li C., He Y., Nicolson S., Hirsch M., Weinberg M. S., Zhang P., Kafri T., & Samulski R. J. (2013). “Adeno-Associated Virus Capsid Antigen Presentation Is Dependent on Endosomal Escape.” J Clin Invest. 123(3):1390-401.
62. Li H., Handsaker B., Wysoker A., Fennell T., Ruan J., Homer N., Marth G., Abecasis G., & Durbin R. (2009). “The Sequence Alignment/Map Format and Samtools.” Bioinformatics. 25(16):2078-9.
63. Li H., Malani N., Hamilton S. R., Schlachterman A., Bussadori G., Edmonson S. E., Shah R., Arruda V. R., Mingozzi F., Wright J. F., Bushman F. D., & High K. A. (2011). “Assessing the Potential for Aav Vector Genotoxicity in a Murine Model.” Blood. 117(12):3311-9.
64. Li Y., Miller C. A., Shea L. K., Jiang X., Guzman M. A., Chandler R. J., Ramakrishnan S. M., Smith S. N., Venditti C. P., Vogler C. A., Ory D. S., Ley T. J., & Sands M. S. (2021). “Enhanced Efficacy and Increased Long-Term Toxicity of Cns-Directed, Aav-Based Combination Therapy for Krabbe Disease.” Molecular Therapy. 29(2):691-701.
65. Lichter-Konecki U., Caldovic L., Morizono H., Simpson K., Ah Mew N., & MacLeod E. (1993). “Omithine Transcarbamylase Deficiency.” Genereviews((R)). M. P. Adam, H. H. Ardinger, R. A. Pagon et al. (eds). Seattle (WA): University of Washington, Seattle.
66. Lipshutz G. S., Gruber C. A., Cao Y.-a., Hardy J., Contag C. H., & Gaensler K. M. (2001). “In Utero Delivery of Adeno-Associated Viral Vectors: Intraperitoneal Gene Transfer Produces Long-Term Expression.” Molecular Therapy. 3(3):284-292.
67. Lock M., Alvira M. R., Chen S. J., & Wilson J. M. (2014). “Absolute Determination of Single-Stranded and Self-Complementary Adeno-Associated Viral Vector Genome Titers by Droplet Digital Pcr.” Hum Gene Ther Methods. 25(2):115-25.
68. Long C., Amoasii L., Mireault A. A., McAnally J. R., Li H., Sanchez-Ortiz E., Bhattacharyya S., Shelton J. M., Bassel-Duby R., & Olson E. N. (2016). “Postnatal Genome Editing Partially Restores Dystrophin Expression in a Mouse Model of Muscular Dystrophy.” Science. 351(6271):400-3.
69. Lowes L. P., Alfano L. N., Arnold W. D., Shell R., Prior T. W., McColly M., Lehman K. J., Church K., Sproule D. M., Nagendran S., Menier M., Feltner D. E., Wells C., Kissel J. T., Al-Zaidy S., & Mendell J. (2019). “Impact of Age and Motor Function in a Phase 1/2a Study of Infants with Sma Type 1 Receiving Single-Dose Gene Replacement Therapy.” Pediatr Neurol. 98:39-45.
70. Maestri N. E., Clissold D., & Brusilow S. W. (1999). “Neonatal Onset Ornithine Transcarbamylase Deficiency: A Retrospective Analysis.” J Pediatr. 134(3):268-72.
71. Magellan. (2021). “Urea Cycle Disorders—Therapeutic Class Review (Tcr).” from https://hhs.texas.gov/sites/default/files/documents/about-hhs/communications-events/meetings-events/dur/mav-2020/mav-2020-durb-agenda-item-4gg.pdf.
72. Manno C. S., Pierce G. F., Arruda V. R., Glader B., Ragni M., Rasko J. J., Ozelo M. C., Hoots K., Blatt P., Konkle B., Dake M., Kaye R., Razavi M., Zajko A., Zehnder J., Rustagi P. K., Nakai H., Chew A., Leonard D., Wright J. F., Lessard R. R., Sommer J. M., Tigges M., Sabatino D., Luk A., Jiang H., Mingozzi F., Couto L., Ertl H. C., High K. A., & Kay M. A. (2006). “Successful Transduction of Liver in Hemophilia by Aav-Factor Ix and Limitations Imposed by the Host Immune Response.” Nat Med. 12(3):342-7.
73. McCarty D. M., Young S. M., Jr., & Samulski R. J. (2004). “Integration of Adeno-Associated Virus (Aav) and Recombinant Aav Vectors.” Annu Rev Genet. 38:819-45.
74. Melnick J. L., Mayor H. D., Smith K. O., & Rapp F. (1965). “Association of 20 Milli-Micron Particles with Adenoviruses.” J Bacteriol. 90(1):271-4.
75. Mendell J. R., Al-Zaidy S., Shell R., Arnold W. D., Rodino-Klapac L. R., Prior T. W., Lowes L., Alfano L., Berry K., Church K., Kissel J. T., Nagendran S., L'Italien J., Sproule D. M., Wells C., Cardenas J. A., Heitzer M. D., Kaspar A., Corcoran S., Braun L., Likhite S., Miranda C., Meyer K., Foust K. D., Burghes A. H. M., & Kaspar B. K. (2017). “Single-Dose Gene-Replacement Therapy for Spinal Muscular Atrophy.” N Engl J Med. 377(18):1713-1722.
76. Miller D. G., Petek L. M., & Russell D. W. (2003). “Human Gene Targeting by Adeno-Associated Virus Vectors Is Enhanced by DNA Double-Strand Breaks.” Molecular and cellular biology. 23(10):3550-3557.
77. Miller D. G., Petek L. M., & Russell D. W. (2004). “Adeno-Associated Virus Vectors Integrate at Chromosome Breakage Sites.” Nat Genet. 36(7):767-73.
78. Nathwani A. C., Reiss U. M., Tuddenham E. G., Rosales C., Chowdary P., McIntosh J., Della Peruta M., Lheriteau E., Patel N., Raj D., Riddell A., Pie J., Rangarajan S., Bevan D., Recht M., Shen Y. M., Halka K. G., Basner-Tschakarjan E., Mingozzi F., High K. A., Allay J., Kay M. A., Ng C. Y., Zhou J., Cancio M., Morton C. L., Gray J. T., Srivastava D., Nienhuis A. W., & Davidoff A. M. (2014). “Long-Term Safety and Efficacy of Factor Ix Gene Therapy in Hemophilia B.” N Engl J Med. 371(21):1994-2004.
79. Nathwani A. C., Tuddenham E. G., Rangarajan S., Rosales C., McIntosh J., Linch D. C., Chowdary P., Riddell A., Pie A. J., Harrington C., O'Beirne J., Smith K., Pasi J., Glader B., Rustagi P., Ng C. Y., Kay M. A., Zhou J., Spence Y., Morton C. L., Allay J., Coleman J., Sleep S., Cunningham J. M., Srivastava D., Basner-Tschakarjan E., Mingozzi F., High K. A., Gray J. T., Reiss U. M., Nienhuis A. W., & Davidoff A. M. (2011). “Adenovirus-Associated Virus Vector-Mediated Gene Transfer in Hemophilia B.” N Engl J Med. 365(25):2357-65.
80. Nelson C. E., Hakim C. H., Ousterout D. G., Thakore P. I., Moreb E. A., Castellanos Rivera R. M., Madhavan S., Pan X., Ran F. A., Yan W. X., Asokan A., Zhang F., Duan D., & Gersbach C. A. (2016). “In Vivo Genome Editing Improves Muscle Function in a Mouse Model of Duchenne Muscular Dystrophy.” Science. 351(6271):403-7.
81. Nguyen G. N., Everett J. K., Kafle S., Roche A. M., Raymond H. E., Leiby J., Wood C., Assenmacher C.-A., Merricks E. P., Long C. T., Kazazian H. H., Nichols T. C., Bushman F. D., & Sabatino D. E. (2021). “A Long-Term Study of Aav Gene Therapy in Dogs with Hemophilia a Identifies Clonal Expansions of Transduced Liver Cells.” Nature biotechnology. 39(1):47-55.
82. Nichols T. C., Whitford M. H., Arruda V. R., Stedman H. H., Kay M. A., & High K. A. (2015). “Translational Data from Adeno-Associated Virus-Mediated Gene Therapy of Hemophilia B in Dogs.” Hum Gene Ther Clin Dev. 26(1):5-14.
83. NORD. (2021). “Ornithine Transcarbamylase Deficiency.” Retrieved Jan. 1, 2022, from https://rarediseases.org/rare-diseases/omithine-transcarbamylase-deficiency/.
84. Pachori A. S., Melo L. G., Zhang L., Loda M., Pratt R. E., & Dzau V. J. (2004). “Potential for Germ Line Transmission after Intramyocardial Gene Delivery by Adeno-Associated Virus.” Biochemical and biophysical research communications. 313(3):528-533.
85. Paulk N. (2020). “Gene Therapy: It's Time to Talk About High-Dose Aav.” Genetic Engineering & Biotechnology News, Jul. 2, 2021, from https://www.genengnews.com/commentarv/gene-therapy-its-time-to-talk-about-high-dose-aav/.
86. Penaud-Budloo M., Le Guiner C., Nowrouzi A., Toromanoff A., Cherel Y., Chenuaud P., Schmidt M., von Kalle C., Rolling F., Moullier P., & Snyder R. O. (2008). “Adeno-Associated Virus Vector Genomes Persist as Episomal Chromatin in Primate Muscle.” J Virol. 82(16):7875-85.
87. Perrin G. Q., Herzog R. W., & Markusic D. M. (2019). “Update on Clinical Gene Therapy for Hemophilia.” Blood. 133(5):407-414.
88. Pfizer. (2019). “Pfizer Presents Initial Clinical Data on Phase lb Gene Therapy Study for Duchenne Muscular Dystrophy (Dmd).” Retrieved May 15, 2021, from https://investors.pfizer.com/investor-news/press-release-details/2019/Pfizer-presents-initial-clinical-data-on-phase-lb-gene-therapy-study-for-duchenne-muscular-dystrophy-DMD/default.aspx.
89. Pipe S., Becka M., Detering E., Vanevski K., & Lissitchkov T. (2019a). “First-in-Human Gene Therapy Study of Aavhu37 Capsid Vector Technology in Severe Hemophilia A.” Blood. 134(Supplement_1):4630-4630.
90. Pipe S., Hay C., Sheehan J. P., Lissitchkov T., Detering E., & Ferrante F. (2021). “First-in-Human Gene Therapy Study of Bay 2599023 in Severe Hemophilia A: Long-Term Safety and Fviii Activity Results.” Molecular Therapy. 29(4):345-346.
91. Pipe S., Leebeek F. W. G., Ferreira V., Sawyer E. K., & Pasi J. (2019b). “Clinical Considerations for Capsid Choice in the Development of Liver-Targeted Aav-Based Gene Transfer.” Mol Ther Methods Clin Dev. 15:170-178.
92. Rajasekaran S., Thatte J., Periasamy J., Javali A., Jayaram M., Sen D., Krishnagopal A., Jayandharan G. R., & Sambasivan R. (2018). “Infectivity of Adeno-Associated Virus Serotypes in Mouse Testis.” BMC biotechnology. 18(1):1-9.
93. Ran F. A., Cong L., Yan W. X., Scott D. A., Gootenberg J. S., Kriz A. J., Zetsche B., Shalem O., Wu X., Makarova K. S., Koonin E. V., Sharp P. A., & Zhang F. (2015). “In Vivo Genome Editing Using Staphylococcus Aureus Cas9.” Nature. 520(7546):186-91.
94. Rangarajan S., Walsh L., Lester W., Perry D., Madan B., Laffan M., Yu H., Vettermann C., Pierce G. F., Wong W. Y., & Pasi K. J. (2017). “Aav5-Factor Viii Gene Transfer in Severe Hemophilia A.” N Engl J Med. 377(26):2519-2530.
95. Rosas L. E., Grieves J. L., Zaraspe K., La Perle K. M. D., Fu H., & McCarty D. M. (2012). “Patterns of Scaav Vector Insertion Associated with Oncogenic Events in a Mouse Model for Genotoxicity.” Mol Ther. 20(11):2098-2110.
96. Samaranch L., Salegio E. A., San Sebastian W., Kells A. P., Bringas J. R., Forsayeth J., & Bankiewicz K. S. (2013). “Strong Cortical and Spinal Cord Transduction after Aav7 and Aav9 Delivery into the Cerebrospinal Fluid of Nonhuman Primates.” Hum Gene Ther. 24(5):526-32.
97. Scartezini M., Hubbart C., Whittall R. A., Cooper J. A., Neil A. H., & Humphries S. E. (2007). “The Pcsk9 Gene R461 Variant Is Associated with Lower Plasma Lipid Levels and Cardiovascular Risk in Healthy U. K. Men.” Clin Sci (Lond). 113(11):435-41.
98. Schuettrumpf J., Liu J.-H., Couto L. B., Addya K., Leonard D. G., Zhen Z., Summer J., & Arruda V. R. (2006). “Inadvertent Germline Transmission of Aav2 Vector: Findings in a Rabbit Model Correlate with Those in a Human Clinical Trial.” Molecular Therapy. 13(6):1064-1073.
99. Schuster D. J., Dykstra J. A., Riedl M. S., Kitto K. F., Belur L. R., McIvor R. S., Elde R. P., Fairbanks C. A., & Vulchanova L. (2014). “Biodistribution of Adeno-Associated Virus Serotype 9 (Aav9) Vector after Intrathecal and Intravenous Delivery in Mouse.” Front Neuroanat. 8:42.
100. Solid Biosciences. (2019). “Solid Biosciences Provides Data Update from Sgt-001 Development Program.” from https://investors.solidbio.com/node/7866/pdf.
101. Srivastava A. & Carter B. J. (2017). “Aav Infection: Protection from Cancer.” Hum Gene Ther. 28(4):323-327.
102. Suzuki K., Tsunekawa Y., Hernandez-Benitez R., Wu J., Zhu J., Kim E. J., Hatanaka F., Yamamoto M., Araoka T., Li Z., Kurita M., Hishida T., Li M., Aizawa E., Guo S., Chen S., Goebl A., Soligalla R. D., Qu J., Jiang T., Fu X., Jafari M., Esteban C. R., Berggren W. T., Lajara J., Nunez-Delicado E., Guillen P., Campistol J. M., Matsuzaki F., Liu G. H., Magistretti P., Zhang K., Callaway E. M., Zhang K., & Belmonte J. C. (2016). “In Vivo Genome Editing Via Crispr/Cas9 Mediated Homology-Independent Targeted Integration.” Nature. 540(7631):144-149.
103. Tabebordbar M., Zhu K., Cheng J. K. W., Chew W. L., Widrick J. J., Yan W. X., Maesner C., Wu E. Y., Xiao R., Ran F. A., Cong L., Zhang F., Vandenberghe L. H., Church G. M., & Wagers A. J. (2016). “In Vivo Gene Editing in Dystrophic Mouse Muscle and Muscle Stem Cells.” Science. 351(6271):407-411.
104. Tsai S. Q., Topkar V. V., Joung J. K., & Aryee M. J. (2016). “Open-Source Guideseq Software for Analysis of Guide-Seq Data.” Nat Biotechnol. 34(5):483.
105. Tsai S. Q., Zheng Z., Nguyen N. T., Liebers M., Topkar V. V., Thapar V., Wyvekens N., Khayter C., Iafrate A. J., Le L. P., Aryee M. J., & Joung J. K. (2015). “Guide-Seq Enables Genome-Wide Profiling of Off-Target Cleavage by Crispr-Cas Nucleases.” Nat Biotechnol. 33(2):187-197.
106. UniQure. (2021). “Uniqure Announces Fda Removes Clinical Hold on Hemophilia B Gene Therapy Program.” Retrieved May 21, 2021, from https://www.2lobenewswire.com/news-release/2021/04/26/2216691/0/en/uniQure-Announces-FDA-Removes-Clinical-Hold-on-Hemophilia-B-Gene-Therapy-Program.html.
107. Vasquez-Loarte T., Thompson J. D., & Merritt J. L., 2nd (2020). “Considering Proximal Urea Cycle Disorders in Expanded Newborn Screening.” Int J Neonatal Screen. 6(4).
108. Wang L., Bell P., Lin J., Calcedo R., Tarantal A. F., & Wilson J. M. (2011). “Aav8-Mediated Hepatic Gene Transfer in Infant Rhesus Monkeys (Macaca mulatta).” Mol Ther. 19(11):2012-20.
109. Wang L., Breton C., Warzecha C. C., Bell P., Yan H., He Z., White J., Zhu Y., Li M., Buza E. L., Jantz D., & Wilson J. M. (2021). “Long-Term Stable Reduction of Low-Density Lipoprotein in Nonhuman Primates Following in Vivo Genome Editing of Pcsk9.” Mol Ther. 29(6):2019-2029.
110. Wang L., Calcedo R., Wang H., Bell P., Grant R., Vandenberghe L. H., Sanmiguel J., Morizono H., Batshaw M. L., & Wilson J. M. (2010a). “The Pleiotropic Effects of Natural Aav Infections on Liver-Directed Gene Transfer in Macaques.” Mol Ther. 18(1):126-34.
111. Wang L., Smith J., Breton C., Clark P., Zhang J., Ying L., Che Y., Lape J., Bell P., Calcedo R., Buza E. L., Saveliev A., Bartsevich V. V., He Z., White J., Li M., Jantz D., & Wilson J. M. (2018). “Meganuclease Targeting of Pcsk9 in Macaque Liver Leads to Stable Reduction in Serum Cholesterol.” Nat Biotechnol. 36(8):717-725.
112. Wang L., Wang H., Bell P., McCarter R. J., He J., Calcedo R., Vandenberghe L. H., Morizono H., Batshaw M. L., & Wilson J. M. (2010b). “Systematic Evaluation of Aav Vectors for Liver Directed Gene Transfer in Murine Models.” Mol Ther. 18(1):118-25.
113. Wang L., Wang H., Bell P., McMenamin D., & Wilson J. M. (2012). “Hepatic Gene Transfer in Neonatal Mice by Adeno-Associated Virus Serotype 8 Vector.” Hum Gene Ther. 23(5):533-9.
114. Wang L., Yang Y., Breton C., Bell P., Li M., Zhang J., Che Y., Saveliev A., He Z., White J., Latshaw C., Xu C., McMenamin D., Yu H., Morizono H., Batshaw M. L., & Wilson J. M. (2020). “A Mutation-Independent Crispr-Cas9-Mediated Gene Targeting Approach to Treat a Murine Model of Omithine Transcarbamylase Deficiency.” Sci Adv. 6(7):eaax5701.
115. Wang L., Yang Y., Breton C. A., White J., Zhang J., Che Y., Saveliev A., McMenamin D., He Z., Latshaw C., Li M., & Wilson J. M. (2019). “Crispr/Cas9-Mediated in Vivo Gene Targeting Corrects Hemostasis in Newborn and Adult Factor Ix-Knockout Mice.” Blood. 133(26):2745-2752.
116. Watanabe S., Kanatsu-Shinohara M., Ogonuki N., Matoba S., Ogura A., & Shinohara T. (2017). “Adeno-Associated Virus-Mediated Delivery of Genes to Mouse Spermatogonial Stem Cells.” Biology of reproduction. 96(1):221-231.
117. Watanabe S., Kanatsu-Shinohara M., Ogonuki N., Matoba S., Ogura A., & Shinohara T. (2018). “In Vivo Genetic Manipulation of Spermatogonial Stem Cells and Their Microenvironment by Adeno-Associated Viruses.” Stem cell reports. 10(5):1551-1564.
118. Wellman J., Federico M., Ozelo M., & High K. (2012). “Results from the Long-Term Follow-up of Severe Hemophilia B Subjects Previously Enrolled in a Clinical Study of Aav2-Fix Gene Transfer to the Liver.” Mol Ther. 20(Suppl1):528.
119. Yang Y., Wang L., Bell P., McMenamin D., He Z., White J., Yu H., Xu C., Morizono H., Musunuru K., Batshaw M. L., & Wilson J. M. (2016). “A Dual Aav System Enables the Cas9-Mediated Correction of a Metabolic Liver Disease in Newborn Mice.” Nat Biotechnol. 34(3):334-8.
120. Zanta-Boussif M. A., Charrier S., Brice-Ouzet A., Martin S., Opolon P., Thrasher A. J., Hope T. J., & Galy A. (2009). “Validation of a Mutated Pre Sequence Allowing High and Sustained Transgene Expression While Abrogating Whv-X Protein Synthesis: Application to the Gene Therapy of Was.” Gene Ther. 16(5):605-19.
121. Zeng W., Tang L., Bondareva A., Honaramooz A., Tanco V., Dores C., Megee S., Modelski M., Rodriguez-Sosa J. R., & Paczkowski M. (2013). “Viral Transduction of Male Germline Stem Cells Results in Transgene Transmission after Germ Cell Transplantation in Pigs.” Biology of reproduction. 88(1):27, 1-9.
122. Zheng Z., Liebers M., Zhelyazkova B., Cao Y., Panditi D., Lynch K. D., Chen J., Robinson H. E., Shim H. S., Chmielecki J., Pao W., Engelman J. A., Iafrate A. J., & Le L. P. (2014). “Anchored Multiplex Pcr for Targeted Next-Generation Sequencing.” Nat Med. 20(12):1479-84.

1. A dual vector system for treating an ornithine transcarbamylase deficiency, the system comprising:
(a) a gene editing AAV comprising a first AAV rh79 capsid and a first vector genome comprising a 5′ ITR, a sequence encoding a meganuclease having the sequence of SEQ ID NO: 3 that targets PCSK9 under control of regulatory sequences that direct expression of the meganuclease in a target cell comprising a PCSK9 gene, and a 3′ ITR; and
(b) a donor AAV vector comprising a second AAV rh79 capsid and a second vector genome comprising: a 5′ITR, a 5′ homology directed recombination (HDR) arm, a transgene comprising the sequence of SEQ ID NO: 4, or a sequence at least 90% identical to SEQ ID NO: 4 encoding ornithine transcarbamylase (OTC) and regulatory sequences that direct expression of the transgene in the target cell, a 3′ HDR arm, and a 3′ ITR.

2. The dual vector system of claim 1, wherein the sequence encoding the meganuclease comprises nucleotides (nt) 1089-2183 of SEQ ID NO: 2, or a sequence at least 90% identical to nucleotides (nt) 1089-2183 of SEQ ID NO: 2.

3. The dual vector system of claim 1, wherein the transgene encoding OTC comprises SEQ ID NO: 4.

4. The dual vector system of claim 1, wherein the first and second AAV capsid are AAVrh79 capsids of SEQ ID NO: 16.

5. The dual vector system of claim 1, wherein the ratio of gene editing AAV vector of (a) to donor AAV vector of (b) is 1:3.

6. The dual vector system of claim 1, wherein the nuclease is under the control of a TBG promoter.

7. The dual vector system of claim 1, wherein the transgene is under the control of a TBG promoter.

8. The dual vector system according to of claim 1, wherein
i) the first vector genome comprises nt 211 to 2964 of SEQ ID NO: 2, or a sequence sharing at least 90% identity with nt 211 to 2964 of SEQ ID NO: 2; and
ii) the second vector genome comprises nt 178 to 3281 of SEQ ID NO: 6 or a sequence sharing at least 90% identity with nt 178 to 3281 of SEQ ID NO: 6.

9. A method of treating an OTC deficiency in humans by co-administering the dual vector system of claim 1.

10. A method of treating an OTC deficiency in a subject, the method comprising: co-administering to the subject having OTC:
(a) a gene editing AAV comprising a first AAV rh79 capsid and a first vector genome comprising a 5′ ITR, a sequence encoding a meganuclease having the sequence of SEQ ID NO: 3 that targets PCSK9 under control of regulatory sequences that direct expression of the meganuclease in a target cell comprising a PCSK9 gene, and a 3′ ITR; and
(b) a donor AAV vector comprising a second AAV rh79 capsid and a second vector genome comprising: a 5′ITR, a 5′ homology directed recombination (HDR) arm, a transgene comprising the sequence of SEQ ID NO: 4, or a sequence at least 90% identical to SEQ ID NO: 4 encoding ornithine transcarbamylase (OTC) and regulatory sequences that direct expression of the transgene in the target cell, a 3′ HDR arm, and a 3′ ITR.

11. The method according to claim 10, wherein
i) the first vector genome comprises nt 211 to 2964 of SEQ ID NO: 2, or a sequence sharing at least 90% identity with nt 211 to 2964 of SEQ ID NO: 2; and
ii) the second vector genome comprises nt 178 to 3281 of SEQ ID NO: 6 or a sequence sharing at least 90% identity with nt 178 to 3281 of SEQ ID NO: 6.

12. The method of claim 9, wherein the gene editing AAV vector of (a) and the donor vector of (b) are delivered essentially simultaneously via IV.

13. The method of claim 9, wherein the gene editing AAV vector of (a) is suspended in a vehicle for injection at a dosage of about 1×10¹³ GC/kg.

14. The method of claim 9, wherein the AAV donor vector of (b) is suspended in a vehicle for injection at a concentration of about 3×10¹³ GC/kg.

15. The method of claim 9, wherein the subject is from age 1 day to 4 months old.

16. The method of claim 9, wherein the subject is male.

17-18. (canceled)
